(19) United States
(12) Patent Application Publication
(10) Pub. No.: US 2023/0003744 A1
(43) Pub. Date: Jan. 5, 2023

(54) CSF-BASED PROGNOSTIC BIOMARKERS IN ALZHEIMER'S DISEASE AND METHODS OF USE THEREOF

(71) Applicants: Rutgers, The State University of New Jersey, New Brunswick, NJ (US); Emory University, Atlanta, GA (US)

(72) Inventor: William T. Hu, New Brunswick, NJ (US)

(21) Appl. No.: 17/842,433

(22) Filed: Jun. 16, 2022

Related U.S. Application Data
(60) Provisional application No. 63/211,800, filed on Jun. 17, 2021.

(51) Int. Cl. G01N 33/68

(52) U.S. Cl. CPC G01N 33/6896; G01N 2800/2821; G01N 2800/56

(57) ABSTRACT

Embodiments of the disclosure are directed to biomarkers, or a panel of biomarkers, that determine progression of Alzheimer's disease, and methods of use thereof.

### CROSS-REFERENCE TO RELATED APPLICATIONS

This application claims the benefit under 35 U.S.C. §119(e) of U.S. Provisional Application No. 63/211,800, filed Jun. 17, 2021, which is incorporated herein by reference in its entirety.

### STATEMENT OF RIGHTS TO INVENTIONS MADE UNDER FEDERALLY SPONSORED RESEARCH

This invention was made with government support under Grant Nos. R01 AG 054046, R21 AG 043885, K01AG042498, and U01 AG024904 awarded by the National Institutes of Health; and Grant No. W81XWH-12-2-0012 awarded by the Department of Defense. The government has certain rights in the invention.

### FIELD OF DISCLOSURE

Embodiments of the present invention are directed to biomarkers of Alzheimer's disease and methods of use thereof.

### BACKGROUND

Alzheimer's disease (AD) is one of several common neurodegenerative disorders with more than 44 million people affected worldwide and an estimated 5.5 million people currently in the United States. AD is a progressive disorder that develops over several years from undiagnosed cellular changes to mild cognitive impairment (MCI) to the conversion to dementia. This process can take anywhere from 5-59 months that makes discussing prognosis and designing therapeutic trials challenging.

Although neuroimaging techniques are typically used to check the progression of AD, early and accurate detection of core Alzheimer's disease (AD) pathologies is increasingly probable with reliable cerebrospinal fluid (CSF) and molecular imaging biomarkers. Longitudinal studies have consistently shown biomarkers for AD neuropathologic changes (ADNC) to address if people with the earliest cognitive symptoms will undergo cognitive decline, but they poorly address when such decline will occur—especially at the individual level (FIG. 2). Several reasons for this include the relatively stable trajectory of CSF (amyloid and tau) or PET amyloid biomarkers once in the symptomatic stages of AD, clinical and pathologic AD heterogeneity, and mixed pathologies. Biomarkers reflecting biological processes commonly found in AD, but relatively independent of the formation of neuritic plaques and neurofibrillary tangles, may provide information on the brain's susceptibility or resistance to ADNC.

Among these processes, neuroinflammation is consistently identified to relate to AD pathogenesis. Multiple genetic variants associated with increased AD risks were found in immune-related genes including CD33, CR1, HLA-DRB5-HLA-DRB1, MEF2C, TREIVI2, and PLCG2. Neuropathologic analysis has commonly shown microglial activation in AD, and stage-specific CSF alterations in complement and interleukin levels have been shown. Moreover, AD appears to specifically modify aging-related T-cell cytokine alterations (also known as, inflammaging). Thus, CSF inflammatory proteins and peptides represent promising candidates to inform rates of AD progression. In keeping with this, higher CSF soluble TREM2 (sTREM2) levels were recently linked to slower AD progression in the Alzheimer's Disease Neuroimaging Initiative (ADNI). Loss-of-function mutations in the sTREM2 sheddace TACE/ADAM17 are linked to AD, and other substrates for this enzyme are may be used in the assessment of AD-related inflammatory changes.

Because CSF inflammatory proteins are regulated by interlinked pro- and anti-inflammatory processes, it is not straightforward to postulate how their levels vary according to each other and AD. Ideal CSF inflammatory biomarkers also have readily available assays with high accuracy and intermediate precision, and CSF changes relatively orthogonal to core AD biomarkers beta-amyloid 1-42 (Ab42), total tau (t-Tau), and tau phosphorylated at threonine 181 (p-Tau₁₈₁).

There is a need to identify new methods and biomarkers to predict the potential progression trajectory of the disease, or prognosis, in order to design an appropriate treatment methodology.

### SUMMARY

One aspect of the disclosure provides a method of treating a subject: (a) detecting in a cerebral spinal fluid (CSF) sample from a subject, a level of at least two biomarkers selected from the group consisting of: sTNFR1, sTNFR2, and sVCAM1; and (b) determining progression of Alzheimer's disease (AD) in the subject; and (c) administering a treatment to the subject based on the progression of AD in the subject. Such methods determine the progression of Alzheimer's disease, where AD comprises: normal cognition, mild cognitive impairment (MCI), or dementia. Another aspect is directed to such method, where the determining of (b) comprises: generating a biomarker score based on the level of the at least two biomarkers, where the biomarker score of 0 or greater the subject has slow progression of Alzheimer's disease (AD), where slow progression is conversion to dementia at a median of 36 months; where the biomarker score of less than 0 indicates fast progression of AD, where fast progression is conversion to dementia at a median of 12-18 months; and the method determines progression of Alzheimer's disease in the subject. Yet a further aspect of the disclosure provides such methods further containing at least one biomarker selected from the group consisting of: beta-amyloid 1-42 (Ab42); total tau (t-Tau); tau phosphorylated at threonine 181 (p-Tau₁₈₁); Transforming Growth Factor 1, 2, and 3 (TGFβ1, TGFβ2, TGFβ3); soluble Intercellular Adhesion Molecule 1 (sICAM1); Tumor Necrosis Factor α (TNFα); Interleukin 6 (IL)-6; IL-7; IL-9; IL-10; IL-12p40; IL-21; and Interferon Gamma-Induced Protein 10 (IP-10), or combinations thereof.

In another aspect, such methods use three biomarkers, where the biomarker score is calculated using the formula: 0.334+0.633×zlog₁₀[biomarker 1]+0.544×zlog₁₀[biomarker 2]—0.230×zlog₁₀[biomarker 3], and biomarker 1 is sTNFR1; biomarker 2 is sVCAM1; and biomarker 3 is sTNFR2, where a biomarker score of 0 or greater the subject has slow progression of Alzheimer's disease (AD), and slow progression is conversion to dementia at a median of 36 months, and where a biomarker score of less than 0 indicates fast progression of AD, and fast progression is conversion to dementia at a median of 12-18 months. One aspect of the disclosure is directed to such methods, where Alzheimer's disease or its progression comprises: normal cognition, mild cognitive impairment (MCI), and/or dementia.

A further aspect provides such a method of treating a subject: generating a biomarker score based on the level of the at least two biomarkers, where the biomarker score of −0.435 or greater the subject has slow progression of Alzheimer's disease (AD), where slow progression is conversion to dementia at a median of 36 months; where the biomarker score of less than −0.435 indicates fast progression of AD, where fast progression is conversion to dementia at a median of 12-18 months; and the method determines progression of Alzheimer's disease in the subject. One aspect of the disclosure is directed to such methods, where Alzheimer's disease or its progression comprises: normal cognition, mild cognitive impairment (MCI), and/or dementia.

In yet a further aspect, the method uses three biomarkers, where the biomarker score is calculated using the formula: 0.101+0.633×zlog₁₀[biomarker 1]+0.544×zlog₁₀[biomarker 2]−0.230×zlog₁₀[biomarker 3], and biomarker 1 is sTNFR1; biomarker 2 is sVCAM1; and biomarker 3 is sTNFR2, where a biomarker score of −0.435 or greater the subject has slow progression of Alzheimer's disease (AD), where slow progression is conversion to dementia at a median of 36 months; where the biomarker score of less than −0.435 indicates fast progression of AD, where fast progression is conversion to dementia at a median of 12-18 months.

Another aspect is directed to a panel of biomarkers for use in determining progression of Alzheimer's disease in a CSF sample from a subject, where the biomarkers are sTNFR1, sTNFR2, and sVCAM1. In other aspects of the disclosure, the panel of biomarkers may further comprise biomarkers selected from the group consisting of: beta-amyloid 1-42 (Ab42); total tau (t-Tau); tau phosphorylated at threonine 181 (p-Tau₁₈₁); Transforming Growth Factor 1, 2, and 3 (TGFβ1, TGFβ2, TGFβ3); soluble Intercellular Adhesion Molecule 1 (sICAM1); Tumor Necrosis Factor α (TNFα); Interleukin 6 (IL)-6; IL-7; IL-9; IL-10; IL-12p40; IL-21; and Interferon Gamma-Induced Protein 10 (IP-10), or combinations thereof.

### BRIEF DESCRIPTION OF FIGURES

FIG. 1 illustrates a relationship between two protein family scores (core AD (FIG. 1a) and sTNFR1-related (FIG. 1B)) and conversion from MCI to dementia. Following principal component analysis, core AD principal component (PC) score corresponding to t-Tau/Aβ42 of 0.39 (predicted ADNC threshold) and sTNFR1 PC score corresponding to no net decline at this core AD score were used to stratify ADNI mild cognitive impairment (MCI) participants (FIG. la). Circle colors represent follow-up time, with converters shown as filled circles and non-converters shown as open circles. Participants with high core AD and low sTNFR1 scores had the shortest median time to conversion, followed by those with high scores in both and those with low core AD score (b, *p=0.014 with dementia diagnosis as outcome; †p=0.007 with clinical dementia rating-sum of boxes score (CDR-SB) ≥4 as outcome).

FIG. 2 shows between-subject variability of cognitive decline in ADNI. Longitudinal CDR-SB changes up to 120 months following CSF collection according to clinical diagnosis of NC (FIG. 2a); MCI (FIG. 2b); and AD dementia (FIG. 2c) shows great between-subject variability even after incorporating predicted ADNC status (CSF tTau/Aβ42 levels relative to 0.39 (Shaw, L. M. et al. Ann Neurol 65, 403-413, doi:10.1002/ana.21610 (2009)).

FIG. 3 demonstrates a relationship between two protein family scores (core AD and sTREM2) and cognitive decline in AD dementia. AD dementia participants with high core AD and sTREM2 scores had slower conversion to a pre-set CDR-SB threshold (1.5 S.D. above the mean) than participants with low sTREM2 scores (FIG. 3a, *p=0.001). Concentrations of two proteins which did (e.g., sTREM2 and progranulin) and one did not (e.g., sTNFR1) load onto sTREM2 score showed greatest group-level differences in sTREM2 (FIG. 3b; median and interquartile ranges shown). Substituting corresponding p-Tau₁₈₁ and sTREM2 concentrations (shown in brackets to distinguish from a) for corresponding scores modestly reproduced the distinction (FIG. 3c, †p=0.028).

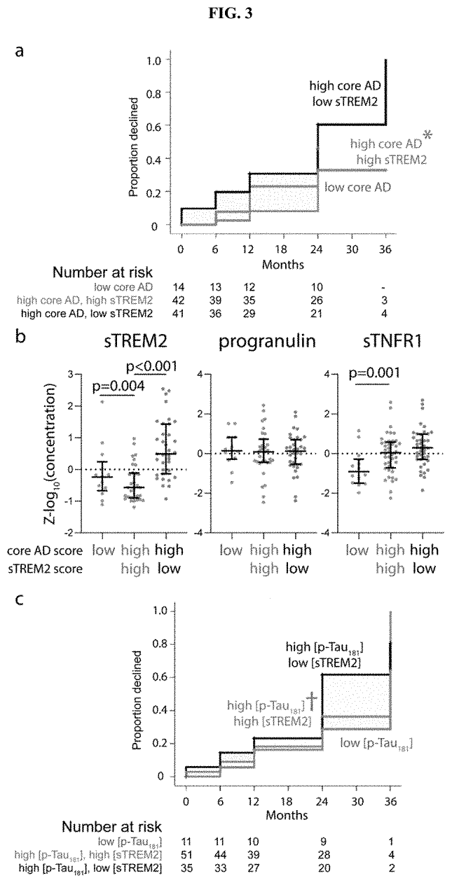

FIG. 4 provides a frequency of baseline CDR-SB scores according to baseline diagnosis. From left to right, respectively, the bars represent NC (left bar; #), MCI (center bar; *), and AD dementia (right bar; ^).

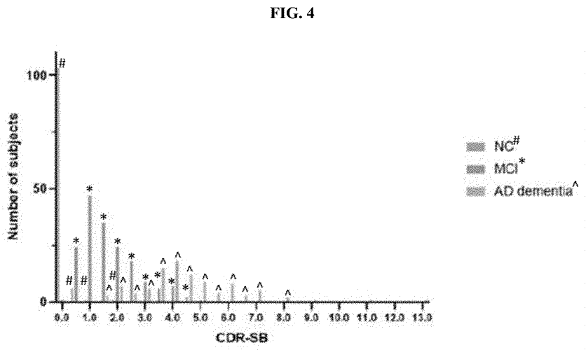

FIG. 5 shows a relationship between two protein family scores (core AD and IL6) and cognitive decline in participants with normal cognition (NC). NC participants with high core AD scores had earlier conversion to diagnosis of mild cognitive impairment (MCI; FIG. 5a, p=0.021) or global clinical dementia rating score (CDR) of 0.5 (FIG. 5b, p=0.023), but further stratification according to IL6 score did not further refine long-term cognitive outcomes. Sample size in the NC cohort was 109 after excluding two participants with very rapid decline from NC to dementia. Low core AD, high IL score (dashed line).

FIG. 6 presents Mild Cognitive Impairment (MCI) conversion in the ADNI and Atlanta-based MCI cohorts. Conversion to dementia diagnosis based on the same p-Tau₁₈₁ levels and a regression-based prediction of sTNFR1 score (γ_{sTNFR1}) reproduced the prognostic profiles from ADNI (FIG. 6a) in the replication cohort (FIG. 6b, n=49). *Lower conversion compared to those with p-Tau₁₈₁>24.1 pg/mL but low Y_{sTNFR1} (p=0.049 in ADNI and p=0.038 in the Atlanta cohort). †Two subgroups with p-Tau₁₈₁ <24.1 were combined due to small numbers, p=0.068 vs. high p-Tau₁₈₁ and low Y_{sTNFR1}. At 24 months, from top to bottom: high pTaum, low Y_{sTNFR1}; high pTau₁₈₁, high Y_{sTNFR1}; low pTau₁₈₁, high Y_{sTNFR1};

### DETAILED DESCRIPTION

Embodiments of the disclosure are directed to biomarkers for identifying the potential progression trajectory of a memory impairment disease (e.g., Alzheimer's disease) in order to design an appropriate treatment methodology.

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%.

By “consist essentially” it is meant that the ingredients include only the listed components along with the normal impurities present in commercial materials and with any other additives present at levels which do not affect the operation of the invention, for instance at levels less than 5% by weight or less than 1% or even 0.5% by weight.

In one embodiment of the disclosure, levels of 15 CSF inflammatory proteins implicated in microglial- and T-cell-mediated processes in the multi-centered Alzheimer's Disease Neuro-imaging Initiative (ADNI, TABLE 1) blinded to diagnosis and prognosis were identified. Another embodiment provides for use of at least two biomarkers selected from CSF inflammatory proteins, but not limited to: soluble Tumor Necrosis Factor Receptor 1 and 2 (sTNFR1, sTNFR2); Transforming Growth Factor 1, 2, and 3 (TGFβ1, TGFβ2, TGFβ3); soluble Intercellular Adhesion Molecule 1 (sICAM1), soluble Vascular Cell Adhesion Molecule 1 (sVCAM1); Tumor Necrosis Factor α (TNFα); Interleukin 6 (IL-6), IL-7, IL-9, IL-10, IL-12p40, IL-21; and Interferon Gamma-Induced Protein 10 (IP-10).

In yet another embodiment of the disclosure, at least two biomarkers may include, but are not limited to, sTNFR1, sTNFR2, sICAM1, sVCAM1, and TNFα, which are all substrates for TACE/ADAM17 (REFs 8,14-18). Some embodiments may be directed to the use of principal component analysis (PCA) within each ADNI diagnostic category for identifying consistently correlated families of proteins, and sTNFR1-related proteins and soluble triggering receptor expressed on myeloid cells 2 (sTREM2) to associate with prognosis (Group, F.-N. B. W. (eds Food and Drug Administration (US) & National institutes of Health (US)) 23-26 (Food and Drug Administration and National Institutes of Health, Silver Spring (Md.) Bethesda (Md.), 2016).) (rates and likelihood of decline) in mild cognitive impairment (MCI) and AD dementia in a diagnosis-specific manner.

One embodiment of the disclosure provides a method of treatment, comprising: (a) detecting in a cerebral spinal fluid (CSF) sample from a subject, a level of at least two biomarkers selected from the group consisting of: sTNFR1, sTNFR2, and sVCAM1; and (b) determining progression of Alzheimer's disease (AD) in the subject; and (c) administering a treatment to the subject based on the progression of AD in the subject. Such methods determine the progression of Alzheimer's disease, where AD comprises: normal cognition, mild cognitive impairment (MCI), or dementia. Another embodiment is directed to such method, where the determining of (b) comprises: generating a biomarker score based on the level of the at least two biomarkers, where the biomarker score of 0 or greater the subject has slow progression of Alzheimer's disease (AD), where slow progression is conversion to dementia at a median of 36 months; where the biomarker score of less than 0 indicates fast progression of AD, where fast progression is conversion to dementia at a median of 12-18 months; and the method determines progression of Alzheimer's disease in the subject. Yet a further embodiment of the disclosure provides such methods further containing at least one biomarker selected from the group consisting of: beta-amyloid 1-42 (Ab42); total tau (t-Tau); tau phosphorylated at threonine 181 (p-Tau₁₈₁); Transforming Growth Factor 1, 2, and 3 (TGFβ1, TGFβ2, TGFβ3); soluble Intercellular Adhesion Molecule 1 (sICAM1); Tumor Necrosis Factor α (TNFα); Interleukin 6 (IL)-6; IL-7; IL-9; IL-10; IL-12p40; IL-21; and Interferon Gamma-Induced Protein 10 (IP-10), or combinations thereof.

In another embodiment, such methods of treatment use three biomarkers, where the biomarker score is calculated using the formula: 0.334+0.633×zlog₁₀[biomarker 1]+0.544×zlog₁₀[biomarker 2]−0.230×zlog₁₀[biomarker 3], and biomarker 1 is sTNFR1; biomarker 2 is sVCAM1; and biomarker 3 is sTNFR2, where a biomarker score of 0 or greater the subject has slow progression of Alzheimer's disease (AD), and slow progression is conversion to dementia at a median of 36 months, and where a biomarker score of less than 0 indicates fast progression of AD, and fast progression is conversion to dementia at a median of 12-18 months. One embodiment of the disclosure is directed to such methods, where Alzheimer's disease or its progression comprises: normal cognition, mild cognitive impairment (MCI), and/or dementia.

A further embodiment provides such a method of treatment, comprising: generating a biomarker score based on the level of the at least two biomarkers, where the biomarker score of −0.435 or greater the subject has slow progression of Alzheimer's disease (AD), where slow progression is conversion to dementia at a median of 36 months; where the biomarker score of less than −0.435 indicates fast progression of AD, where fast progression is conversion to dementia at a median of 12-18 months; and the method determines progression of Alzheimer's disease in the subject. One embodiment of the disclosure is directed to such methods, where Alzheimer's disease or its progression comprises: normal cognition, mild cognitive impairment (MCI), and/or dementia.

In yet a further embodiment, the methods of treatment uses three biomarkers, where the biomarker score is calculated using the formula: 0.101+0.633×zlog₁₀[biomarker 1]+0.544×zlog₁₀[biomarker 2]−0.230×zlog₁₀[biomarker 3], and biomarker 1 is sTNFR1; biomarker 2 is sVCAM1; and biomarker 3 is sTNFR2, where a biomarker score of −0.435 or greater the subject has slow progression of Alzheimer's disease (AD), where slow progression is conversion to dementia at a median of 36 months; where the biomarker score of less than −0.435 indicates fast progression of AD, where fast progression is conversion to dementia at a median of 12-18 months.

Another embodiment is directed to a panel of biomarkers for use in determining progression of Alzheimer's disease in a CSF sample from a subject, where the biomarkers are sTNFR1, sTNFR2, and sVCAM1. In other embodiments of the disclosure, the panel of biomarkers may further comprise biomarkers selected from the group consisting of: beta-amyloid 1-42 (Ab42); total tau (t-Tau); tau phosphorylated at threonine 181 (p-Tau₁₈₁); Transforming Growth Factor 1, 2, and 3 (TGFβ1, TGFβ2, TGFβ3); soluble Intercellular Adhesion Molecule 1 (sICAM1); Tumor Necrosis Factor α (TNFα); Interleukin 6 (IL)-6; IL-7; IL-9; IL-10; IL-12p40; IL-21; and Interferon Gamma-Induced Protein 10 (IP-10), or combinations thereof.

In older people with memory loss, levels of CSF Alzheimer's disease (AD)-related proteins (Aβ42, t-Tau, p-Tau₁₈₁) and their ratios (t-Tau/Aβ42) can be used to determine if AD is the etiology. CSF levels of other biomarker levels may be measured, where the biomarker may be selected from, but not limited to, TNFα, sTNFR1, sTNFR2, sVCAM1, sICAM1, IP-10, TGF-β1, TGF-β2, TGF-β3, IL-6, IL-7, IL-9, IL-10, IL-12p40, IL-21 sTREM2, and progranulin. Based on the methods disclosed here, the biomarkers may be separated into 7 protein clusters/families (see, e.g., TABLE 2). Proteins within each cluster/family differentially contribute to a family principal component (PC) score.

The sTNFR1 family includes sTNFR1, sTNFR2, sVCAM1, TGF-β1, sICAM1, and IP-10. In one embodiment, the biomarkers used in the methods of the disclosure may be selected from those in the sTNFR1 family.

In people with very mild memory loss or mild cognitive impairment due to underlying Alzheimer's disease, the sTNFR1 family PC score separates people into fast progressors (conversion to dementia at a median of 12-18 months) or slower progressors (conversion to dementia at a median of 36 months).

Such methods of treatment as disclosed may be directed to various treatments, including therapies, of Alzheimer's disease. A clinician or physician treating a subject for Alzheimer's disease understands how to select a course of treatment and type, dosage, the administration amount, length of treatment, and the like, and understands that the type, dosage, and/or length of treatment may periodically be adjusted on the basis of the individual, based on, for example, the individual's weight, age, gender, status, and course of Alzheimer's disease including related symptoms, whether prophylactic or therapeutic, and other factors that affect the treatment (e.g., drug delivery, absorption, pharmacokinetics or half-life, and pharmacological effects). Non-limiting examples of Alzheimer's disease treatment in a subject include use or administration of: Cholinesterase inhibitors (e.g., galantamine, rivastigmine, donepezil); inhibitors of acetylcholine and/or butyrylcholine breakdown (e.g., donepezil, rivastigmine); N-methyl D-aspartate (NMDA) antagonist (e.g., memantine); glutamate regulators (e.g., memantine); nicotinic receptor stimulators (e.g., galantamine); amyloid beta plaque reducers (e.g., aducanumab); or combinations thereof.

### EXAMPLES

The following examples illustrate specific aspects of the instant description. The examples should not be construed as limiting, as the example merely provides specific understanding and practice of the embodiments and its various aspects.

### Example 1: Ethics Approval

This study was approved by the Emory University Institutional Review Board, the National Institutes on Aging, and the ADNI Resource Allocation and Review Committee. The ADNI was launched in 2003 as a public-private partnership, led by Principal Investigator Michael W. Weiner, M D. The primary goal of ADNI has been to test whether serial magnetic resonance imaging, positron emission tomography, other biological markers, and clinical and neuropsychological assessment can be combined to measure the progression of MCI and early AD dementia. All participants or legally-authorized representatives provided written informed consent to participate in the studies.

### Example 2: Study Design and Participants

A cross-sectional study design was used for PCA of CSF AD and inflammatory proteins. ADNI participants (NCT00106899) were selected to have adequate representation of healthy control participants with normal cognition (NC; n=111) to establish normative range of inflammatory proteins; sufficient numbers of MCI (n=174) and AD dementia (n=97) to identify CSF proteins associated with rates of longitudinal cognitive and functional decline; maximize overlap with participants with measured levels of complement 3 and complement factor H (Hu, W. T. et al. Acta Neuropathol Commun 4, 14, doi:10.1186/s40478-016-0277-8 (2016)); and match sample availability. A power calculation was performed to arrive at a total sample size of at least 250 (MCI and AD dementia together) to have a power of 0.95 to detect an effect size of 0.15 for fixed factors (sex, APOE ε4 status) and covariates (age, baseline cognitive function, two inflammatory protein levels, two core AD biomarker levels) in LMM of longitudinal cognitive decline with p=0.05 if the repeated measure correlation is 0.3, and has power of 0.83 to detect an effect size of 0.15 with p=0.02 if the repeated measure correlation is 0.2.

Other than ADNI participants, two other previously published cohorts were included for replication of the relationship among CSF biomarkers (EXAMPLE 9; TABLE 12). Cohort B (NCT02089555; PI: WTH) was a cohort of older white (n=68) and Black American (n=62) participants with normal cognition (NC), mild cognitive impairment (MCI), and Alzheimer's disease (AD) dementia recruited from Georgia who underwent detailed prospective baseline neurological, neuropsychological, CSF, and MRI characterization for identifying race-associated biomarker differences (Howell, J. C. et al. Alzheimers Res Ther 9, 88, doi:10.1186/s13195-017-0315-1 (2017)). Participants in this study were recruited from the Emory Cognitive Neurology Clinic, Emory Alzheimer's Disease Research Center, or community events in the greater Atlanta area. Cohort C (NCT00135226; PI: WW) was a cohort of middle-aged to older white (n=47) and Black participants with NC (n=21) who underwent detailed prospective baseline and longitudinal neuropsychological, CSF, and MRI characterization for identifying effect of race on vascular and AD biomarkers (Wharton, W. et al. Ann Neurol 86, 407-418, doi:10.1002/ana.25543 (2019); Kumar, V. V. et al. J Alzheimers Dis 75, 109-117, doi:10.3233/JAD-191103 (2020)). Participants in this study were recruited from the Emory Alzheimer's Disease Research Center, community events, or dementia caregiving groups in the greater Atlanta. Because of race-based differences in CSF tau-related and inflammatory protein levels (Wharton, W. et al. Ann Neurol 86, 407-418, doi:10.1002/ana.25543 (2019); Howell, J. C. et al. Alzheimers Res Ther 9, 88, doi:10.1186/s13195-017-0315-1 (2017); Morris, J. C. et al. JAMA Neurol, doi:10.1001/jamaneurol.2018.4249 (2019)), only white participants from Cohorts B & C (n=115) were used to replicate PCA findings from ADNI.

To validate the relationship between p-Tau₁₈₁-sTNFR1 combination and longitudinal decline in MCI, an MCI validation cohort (n=49) was created by combining white and Black MCI participants from Cohort B (n=33) who underwent longitudinal follow-up and a separate group of older white and Black MCI participants (n=16) who underwent similar longitudinal clinical and neuropsychological evaluations following detailed baseline neurological, neuropsychological, CSF, and MRI analysis.

### Example 3: CSF Biomarker Measurements

For ADNI, blinded CSF samples were shipped from the Biomarker Core (University of Pennsylvania) to Emory University for analysis in 2018. CSF analysis was performed by two skilled research scientists experienced in multiplex assays blinded to diagnosis and other subject-level information. All samples were run in duplicate with six CSF standards on each plate, and CSF inflammatory protein levels were normalized across plates using the six CSF standard values. ADNI CSF samples were first randomized across twelve 96-well plates, and each batch was analysed for levels of all 15 proteins during the same two-day block to avoid freeze-thawing: Assay 1 included sTNFR1 and sTNFR2; assay 2 included TGFβ 1,2, and 3; assay 3 included IL-21; assay 4 included sICAM-1 and sVCAM-1; and assay 5 included TNFα, IL-6, IL-7, and IL-10 for the first 98 participants, and TNFα, IL-6, IL-7, IL-10, IL-9, IL-12p40, and IP-10 for the remaining 280 participants (see Missing Data). All samples were run in duplicate with six CSF standards on each plate. CSF inflammatory protein levels were normalized across plates using the six CSF standard values, and intermediate precision for each analyte was then calculated using inter-plate coefficient of variation (CV): 9.38% for TNFa, 2.85% for sTNFR1, 3.09% for sTNFR2, 10.99% for sVCAM1, 9.86% for sICAM1, 6.30% for IL-6, 14.68% for IL-7, 9.24% for IL-9, 16.51% for IL-10, 6.41% for IL-12p40, 20.6% for IL-21, 4.83% for IP-10, 8.62% for TGFb1, 7.62% for TGFb2, 7.70% for TGFb3. Values for Ab42, t-Tau, p-Tau₁₈₁, sTREM2 (from MSD and Washington University [WU]) (Ewers, M. et al. Sci Trans! Med 11, doi:10.1126/scitranslmed.aav6221 (2019)), and soluble progranulin levels were obtained from ADNI. CSF biomarkers for the three Atlanta cohorts were analysed by the same laboratory research scientists using the same volume, CSF standards, blinding, and randomization scheme (Wharton, W. et al. Ann Neurol 86, 407-418, doi:10.1002/ana.25543 (2019); Howell, J. C. et al. Alzheimers Res Ther 9, 88, doi:10.1186/s13195-017-0315-1 (2017)).

### Example 4: CSF Inflammatory Proteins Form Reproducible Families Across Diagnostic Categories

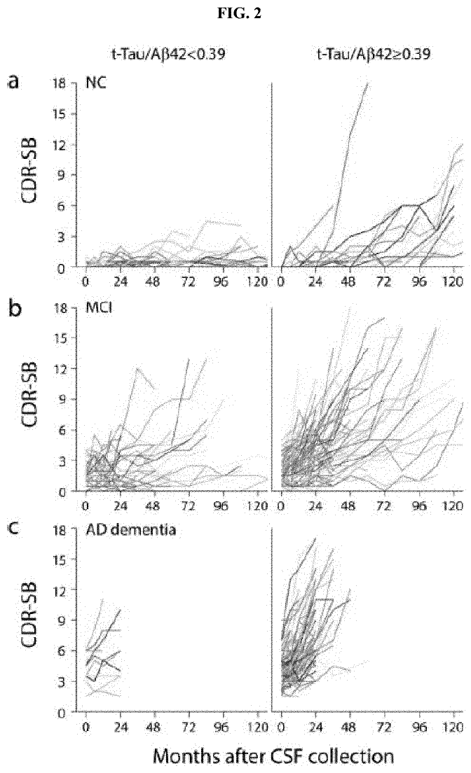

ADNI participants included in the study showed highly variable rates of longitudinal cognitive and functional decline even when classified by clinical diagnosis and predicted ADNC status (by t-Tau/Aβ42 ratio; FIG. 2, TABLE 3) (Shaw, L. M. et al. Ann Neurol 65, 403-413, doi:10.1002/ana.21610 (2009)). To identify potential correlations between functionally-related CSF inflammatory proteins (after log-10 and Z-transformation; see EXAMPLES 1-3, 10-11 and TABLE 4 and TABLE 5), dimension reduction was performed using PCA (Ringner, M. Nat Biotechnol 26, 303-304, doi:10.1038/nbt0308-303 (2008)) on the 15 proteins with previously measured levels of core AD biomarkers (Aβ42, t-Tau, p-Tau₁₈₁, t-Tau/Aβ42), sTREM2, and progranulin. PCA was conducted independently within the normal cognition (NC), MCI, and AD dementia cohort to avoid false discovery due to contrast between extreme subgroups (NC vs moderate AD dementia). Six highly reproducible principal components (PCs) were identified within each diagnostic category (TABLE 2 and TABLE 6): core AD, sTNFR1 (also sTNFR2, sVCAM1, sICAM1, TGFβ1), sTREM2, IL-6/IL-10, TGFβ (TGFβ2, TGFβ1), and IL-7/TNF-α, even though there were instances where some variables demonstrated diagnosis-specific loading (e.g., IP-10 on PC2 in the two groups with AD dementia). People with NC and MCI additionally shared another PC consisting of IP-10 and IL-12p40. PCA in two independent cohorts for whom core AD biomarkers and nine CSF inflammatory protein levels were previously and independently measured (see, e.g., EXAMPLE 9; TABLE 12) replicated PCs for core AD, sTNFR1, IL-10, and IL-7/TNF-α PCs, but sTREM2 (measured using a commercially available assay distinct from the two used in ADNI) became a member of the sTNFR1 PC. These highly reproducible groupings provided strong empirical evidence for CSF inflammatory biomarker families or clusters.

### Example 5: CSF AD and Pro-Inflammatory Alterations each Associated with 5-Year Cognitive Decline in MCI

Based on the inflammatory protein PCs' orthogonality from each other and from core AD biomarker PC, PC scores were tested to determine if they correlated with rates of longitudinal cognitive and functional decline. Analyses were initiated with MCI participants because they had longer follow-up duration than AD dementia and higher likelihood of decline than NC. Linear mixed modeling (LMM)—taking into account baseline characteristics (age, sex, self-reported race, APOE genotype) and CSF protein family PC scores—was paired with two measures of decline: average of composite ADNI Memory and Executive Function scores (ADNI-Mem-EF) and Clinical Dementia Rating Sum of Boxes (CDR-SB). Models which incorporated non-AD biomarker scores outperformed those which only incorporated core AD scores (TABLE 7, TABLE 8), with faster decline during 60 months following CSF collection independently associated with older age (p<0.001), greater core AD score (p<0.001), and lower sTNFR1 score levels (p<0.002, TABLE 9) for both outcomes.

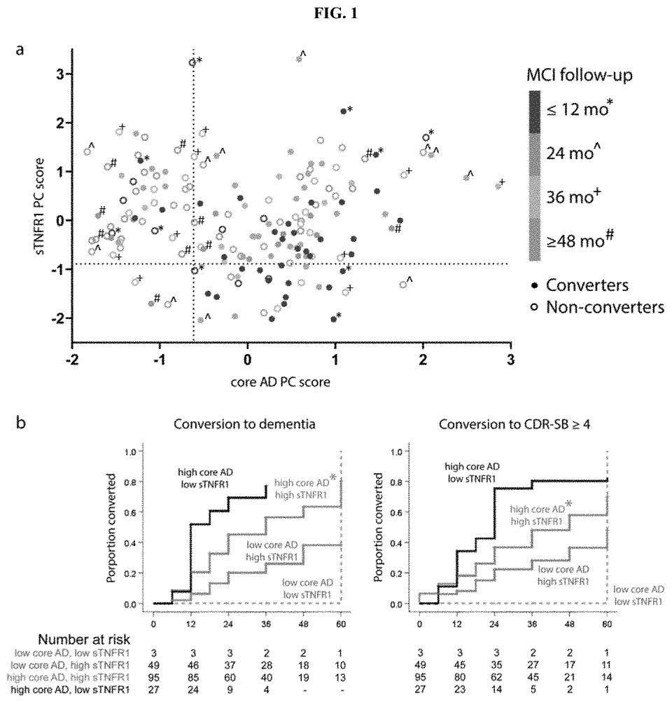

To test whether specific biomarker PC scores can risk stratify within the MCI cohort, the MCI group was first split into those with high or low core AD biomarker scores according to the value (−0.614) corresponding to the t-Tau/Aβ42 threshold for ADNC (Shaw, L. M. et al. Ann Neural 65, 403-413, doi:10.1002/ana.21610 (2009)). At this core AD score threshold, a sTNFR1 score of -0.890 was expected to result in no net decline in ADNI-Mem-EF over time and was therefore used to further stratify MCI participants (FIG. la). Participants with high core AD score and low sTNFR1 score were found to have the earliest decline (according to diagnostic conversion or CDR-SB) during the 60 months following CSF collection. Compared to this group, those with similar core AD scores but higher sTNFR1 scores were less likely to decline (p=0.014 by consensus diagnosis, median time to conversion of 36 vs.12 months; p=0.007 by CDR-SB≥4, median time to conversion of 48 vs.24 months, FIG. 1b). Because dementia diagnosis and greater CDR-SB both imply functional decline beyond worse longitudinal cognitive trajectory (as measured by ADNI-Mem-EF), these findings were interpreted to support the role of sTNFR1 score as a prognostic biomarker in those with MCI due to predicted ADNC.

Two other findings suggested better prognosis associated with higher sTNFR1 scores. First, MCI participants with low core AD score and high sTNFR1 score were even less likely to decline (p<0.001 by diagnostic conversion and p=0.023 by CDR-SB>4 vs high AD score and high sTNFR1 score, median time to conversion of >60 months for both) than participants with two high scores. What's more, only 3 (6%) of 52 MCI participants with low AD scores had low sTNFR1 scores (vs. 22% among those with high AD scores, p=0.005). Cox proportional hazard analysis in all MCI participants with high core AD scores found high sTNFR1 scores to halve the risks of cognitive/functional decline whether assessed by diagnostic conversion (hazards ratio=0.541, 95% CI 0.314-0.933; p=0.027) or CDR-SB (hazards ratio=0.454, 95% CI 0.265-0.778, p=0.004).

### Example 6: CSF sTREM2 Associated with Cognitive Decline in Dementia Stage of AD

The relationship between cognitive decline and baseline CSF biomarker scores in those with AD dementia were analyzed, also using ADNI-Mem-EF and CDR-SB as longitudinal outcome. In this group with greater likelihood of predicted ADNC, core AD score was less correlated with rates of cognitive decline than in MCI. Instead, sTREM2 score was inversely associated with rates of decline (TABLE 10). Using a similar stratification strategy as MCI resulted in too few people with low core AD or sTREM2 score. Therefore, people with high core AD score were divided according to the median sTREM2 score (n=41 for at/above and n=42 for below score of −0.0689), with a smaller third group having low core AD scores (n=14). In keeping with outcomes from MLL analysis, lower sTREM2 score was associated with faster conversion to dementia among those with high core AD scores (median 24 vs. >36 months, p=0.001, FIG. 2a). After adjusting for age, sex, APOE ε4, and baseline CDR-SB, greater sTREM2 scores were associated with reduced risks for decline (H.R.=0.412, 95% CI 0.193-0.878, p=0.022).

Since sTNFR1 score did not translate into a prognostic marker for AD dementia, levels of sTNFR1 among the three AD dementia groups were examined. Whereas sTREM2 levels expectedly differed between those with high and low sTREM2 scores, levels of sTNFR1 and progranulin—another protein loading onto the same sTNFR1 PC—did not (FIG. 2b). Substituting p-Tau181 and sTREM2 concentrations for PC scores modestly reproduced the profiles of decline (median 24 vs. 36 months, p=0.028, FIG. 2c). Therefore, previous findings that greater sTREM2 levels associated with a more benign course of AD dementia were confirmed.

### Example 7: CSF IL6 Associated with Cognitive Change in NC but do not Influence MCI Risks

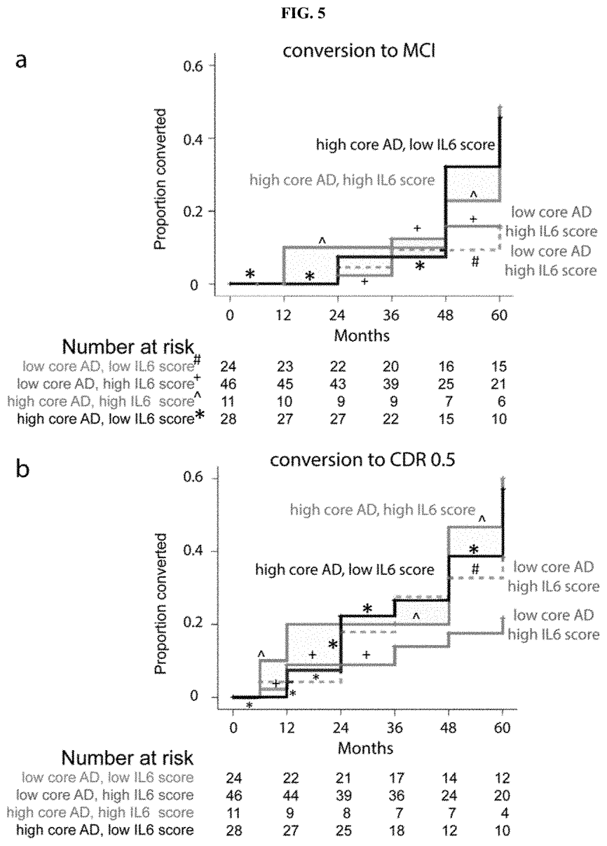

Only cognitive changes in ADNI-Mem-EF were analyzed in NC as most (70 out of 104) had global CDR of 0 at the last follow-up visit (median of 60 months). LMM found baseline cognitive performance, age, and IL6 score each associated with rates of cognitive change through quadratic relationships, while core AD score had a more straightforward linear relationship with cognitive decline (TABLE 11). Stratifying NC participants according to core AD scores showed greater conversion to MCI (p=0.021, FIG. 5a) or CDR 0.5 (p=0.023, FIG. 5b) during the 60 months following CSF collection. Further division according to IL6 scores did not provide additional information on conversion risks most likely due to the overall low rates of decline.

### Example 8: Validation of CSF sTNFR1 as a Prognostic Biomarker in MCI Due to Predicted ADNC

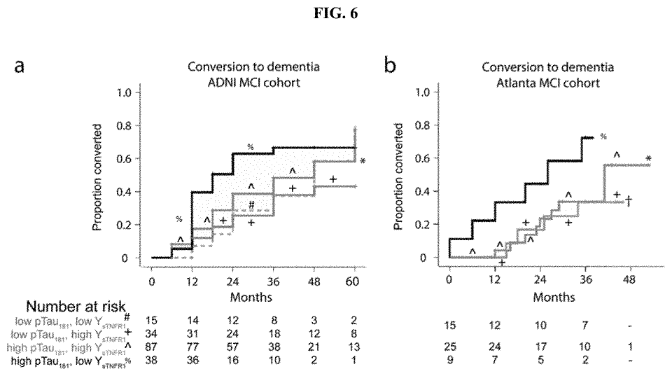

Because individual protein levels are easier to translate as biomarkers in research and clinical settings than protein family scores, the relationship between the top prognostic biomarker scores for MCI and their constituent proteins were also examined to further validate in an independent cohort. Linear regression analysis in the ADNI MCI cohort mapped the core AD score of −0.614 onto z-transformed log₁₀(p-Tau₁₈₁) value of 0.05 or p-TauTau₁₈₁ level of 24.1 pg/mL. On the other hand, sTNFR1 score only modestly associated with sTNFR1 scores despite the high correlation. A regression-based prediction for sTNFR1 score was therefore derived using linear combinations of sTNFR1, sTNFR2, and sVCAM1 levels (all log10- and z-transformed). When paired with p-Tau₁₈₁ levels, this predicted score (ysTNFR1=−0.101+0.633×zlog₁₀[sTNFR1]+0.633×zlog10[sVCAM1]−0.230×zlog10[sTNFR2] performed better in separating MCI participants into groups of different conversion rates than sTNFR1 levels alone (FIG. 6a).

The threshold values for p-Tau₁₈₁ and regression-predicted sTNFR1 score in an independent cohort of 49 MCI participants recruited was applied and longitudinally characterized in Atlanta, including 33 participants from Cohort B in the cross-sectional PCA. Compared to the ADNI MCI participants, these MCI participants were younger (69.3 vs. 75.2, p<0.0001), more likely to have self-reported race as Black (29% vs. 2%, p<0.0001), and had lower CSF t-Tau and p-Tau₁₈₁ levels (TABLE9). These MCI participants as well as their corresponding NC participants also had lower CSF sTNFR1 levels than ADNI participants with the same diagnosis (MCI: mean 645 vs. 904 pg/mL; NC: mean 587 vs. 836 pg/mL) which could result from pre-analytical CSF processing (Gangishetti, U. et al. Alzheimers Res Ther 10, 98, doi:10.1186/s13195-018-0426-3 (2018)) (e.g., ADNI samples had one freeze-thaw cycle before aliquoting), but the NC-based z-transformation accounted for this systemic difference (Gangishetti, U. et al. Alzheimers Res Ther 10, 98, doi:10.1186/s13195-018-0426-3 (2018)). Compared to those with high p-Tau₁₈₁ levels and low predicted TNFR1 score, MCI participants with high p-TauTau₁₈₁ and predicted sTNFR1 scores (p=0.038) or low p-TauTau₁₈₁ levels (p=0.068) each had reduced likelihood of conversion to dementia (FIG. 6b).

### Example 9: Analysis Months After CSF Collection

Cohort B (NCT02089555; PI: WTH) sought to recruit older White and Black American participants with NC, MCI, and AD dementia in Georgia. Inclusion criteria included: age 60-85 (inclusive); has normal cognition, a diagnosis of mild cognitive impairment, or a diagnosis of Alzheimer's disease; self-reported race of Black/African American or non-Hispanic White; able to undergo neuropsychological testing, lumbar puncture, and MRI; and English speaking. Exclusion criteria include: history of large territory stroke; diagnosis of Parkinson's disease, amyotrophic lateral sclerosis, or another progressive neurological disorder which may spare cognition; Mini-Mental State Examination score <17. See, TABLE 12.

Cohort C (NCT00135226; PI: WW) sought to recruit middle-aged to older White and Black participants with NC and family history of dementia in Georgia. Inclusion criteria included: age 45 — 65 (inclusive); a biological parent with AD dementia; willing to fast for eight hours; willing to undergo all procedures including LP. Exclusion Criteria: contraindication for LP; history of neurologic disease or significant head trauma; major untreated depression within two years; history of alcohol or substance abuse; any significant systemic illness or unstable medical condition which could affect cognition or cause difficulty complying with the protocol; diagnosis of MCI, AD dementia, or residence in a skilled nursing facility; use of investigational medication; unwillingness to fast. See, TABLE 12.

### Example 10: Missing Data

Among 382 ADNI participants whose CSF samples were available for this study, the first 100 samples (26%) had 12 proteins measured (all except IL-9, IL-12p40, and IP-10). The remaining 280 samples (74%) had all 15 CSF inflammatory biomarker measured. The 100 samples were from participants randomized across the three diagnostic categories (25 NC, 53 MCI, 22 AD dementia), and they were similar to the remaining participants according to age, sex, APOE ε4 status, and predicted ADNC within each diagnostic category.

319 of the 382 (83%) previously had sTREM2 and progranulin levels measured, with a core group of 241 participants (i.e., 75 NC, 104 MCI, 62 AD dementia) having all CSF proteins (AD biomarkers, sTREM2, progranulin, and inflammatory proteins reported here) measured. PCA was first performed by excluding participants with at least one missing value (listwise), and then with missing values imputation by means and expectation maximization (see EXAMPLE 11).

### Example 11: Statistical Analysis

Data used here—other than levels of 15 CSF inflammatory proteins—were obtained from the ADNI database (adni.loni.usc.edu). All analyses were performed in IBM SPSS 26.0 (Armonk, N.Y.). Two sided tests were used for all analyses. For CSF inflammatory proteins, outliers were determined as log₁₀- and z-transformed values greater than 4 or less than −4. Because individuals who had consistent outlier protein levels were not detected, no outlier inflammatory protein levels were excluded. However, two NC participants had atypical profile of functional decline (FIG. 2) and were excluded from LMM analysis. Two-tailed tests were used in all statistical analysis. CSF inflammatory protein levels were assessed for their normality using Kolmogorov-Smirnov test. All markers except MSD-sTREM2 showed non-normal distribution and were logio transformed (as were t-Tau and p-TauTau₁₈₁ for their skewed distribution). Because mean inflammatory protein levels ranged from 1.10 pg/mL (IL12-p40) to 41.29 ng/mL (sVCAM1), z-transformation of their levels for better assessment of their association with rates of decline relative to each other according to mean and standard deviation (S.D.) values for NC participants at baseline (TABLE 4) was performed.

Analysis of co-variance (ANCOVA) was first used to determine inflammatory protein differences across diagnostic categories and predicted ADNC status, adjusting for age, sex, and APOE ε4 status (TABLE 5). All participants with available measures were included in the ANCOVA for each protein. To identify orthogonal eigenvectors to reduce the number of protein variables and align correlated measures with shared variance, PCA with varimax rotation was then used to separate proteins into PCs using eigenvalues >0.7 as recommended by Jolliffe (Jolliffe, I. T. & Cadima, J. Philos Trans A Math Phys Eng Sci 374, 20150202, doi:10.1098/rsta.2015.0202 (2016)). This was first performed in participants with all protein values available (n=241) and then with missing values replaced with means (values for MCI participants are shown in TABLE 6). Factor loading >0.400 in both missing data handling methods were considered consistent elements of each PC for inclusion in TABLE 2.

Because excluding cases or replacing with means can bias the outcomes (von Hippel, P. T. Amer Statistician 58, 160-164 (2004)), PCA results were additionally confirmed with imputation through expectation maximization to reduce bias (Malan, L., Smuts, C. M., Baumgartner, J. & Ricci, C. Nutr Res 75, 67-76, doi:10.1016/j.nutres.2020.01.001 (2020); Graham, J. W. Annu Rev Psychol 60, 549-576, doi:10.1146/annurev.psych.58.110405.085530 (2009); Dempster, A. P., Laird, N. M. & Rubin, D. B. J Roy Stat Soc B 39, 1-38 (1977)). In the ADNI MCI cohort, this produced identical membership (loading≥0.400) for PC1, PC2, PC3, PC4, PCS, PC6, and PC8, with PC7 now having additional loading by IL-10, TNFa and TGFb3. Thus, missing data handling through three separate approaches all generated highly reproducible PCs.

LMM was used to identify potential predictors of future cognitive decline. Because PCA consistently placed two of the three proteins not measured in all participants (IP-10, IL12-p40) in the same PC, LMM first only focused on participants with measured IP-10 and IL12-p40 levels, but then all participants if IP-10/IL-12 score did not associate with rates of cognitive or functional decline at p<0.10. Cognitive decline in ADNI was analyzed using validated composite Memory (ADNI-Mem) and Executive Function (ADNI-EF) scores previously generated from subtests targeting respective functions through item-response theory and bi-factor confirmatory factor analysis to optimize these scores for longitudinal tracking (Gibbons, L. E. et al. Brain Imaging Behav 6, 517-527, doi:10.1007/s11682-012-9176-1 (2012); Crane, P. K. et al. Brain Imaging Behav 6, 502-516, doi:10.1007/s11682-012-9186-z (2012)). ADNIMem subtests included Rey Auditory Visual Learning Test (learning & recall), AD Assessment Scale—Cognitive Subscale (ADAS-Cog), word recall from Mini-Mental State Examination, and Logical Memory I and II from the Wechsler Memory-Test Revised. ADNI-EF subtests included category fluency, oral Trail Making Test A & B, Digit Span Backwards, Digit Symbol Substitution Test from the Wechsler Adult Intelligence Scale Revised, and Clock Drawing Test. Each factor was normalized to have a mean of 0 and standard deviation of 1, equivalent to a z-score transformation. Because CDR-SB is used to track longitudinal cognitive performance (Williams, M. M., Storandt, M., Roe, C. M. & Morris, J. C. Alzheimers Dement 9, S39-44, doi:10.1016/j jalz.2012.01.005 (2013)) but was not included in ADNI-Mem or ADNI-EF, cognitive decline in ADNI was also analyzed according to CDR-SB.

In LMM, time (in months), Time), and Time (Toledo, J. B., Xie, S. X., Trojanowski, J. Q. & Shaw, L. M. Acta Neuropathol 126, 659-670, doi:10.1007/s00401-013-1151-4 (2013)) were included as fixed and random variables to determine if linear, quadratic, and cubic models best described longitudinal cognitive decline. Akaike Information Criterion (AIC) used to assess whether a model incorporating additional factors (e.g., Time (Montine, T. J. et al. Acta neuropathologica 123, 1-11, doi:10.1007/s00401-011-0910-3 (2012)), core AD score, and interaction terms) was better than a simpler model without overfitting. This created the baseline models without additional PCs. Models incorporating Time only achieved substantially better AIC (Δ>>10) than models additionally incorporating Time (Ref) 2 and Time (Ref)3 terms for MCI and AD dementia, but higher order terms improved the fit for long-term cognitive decline in NC. Subsequent models incorporating Time, demographic variables, and biomarker PC scores were assessed using criteria of substantial (ΔAIC>10), moderate (7≥ΔAIC≥4), or minimal (2>ΔAIC) improvement. For each diagnostic category, ADNI-Mem-EF and CDR-SB were each used as the time-dependent variable of cognitive outcome. PC scores were tested in a stepwise fashion to determine each score's impact on AIC.

To illustrate prognostic impact of CSF biomarker PC scores, KM survival analysis was used to determine their relationship to cognitive decline. Significant cognitive decline in MCI was assessed by diagnostic conversion (to dementia) as well as CDR-SB. Based on prior data from 792 participants longitudinally followed at the Knight AD Research Center at Washington University, 1 standard deviation above the mean for MCI and 1 standard deviation below the mean for AD dementia both had CDR-SB of 3.8.48 Therefore, CDR-SB >4 was selected for MCI participants for significant worsening. Cox-proportional hazard analysis was further used among ADNI MCI participants to determine impact of CSF biomarker PC score on conversion, adjusting for age, sex, and APOE genotype. Core AD biomarker and sTNFR1 scores were initially used in these analyses, but simplified measures more suitable for clinical application were also derived. Linear regression analysis was first used to determine the p-Tau₁₈₁ concentration (in pg/mL) corresponding to the threshold core AD biomarker. Linear regression was also used to determine sTNFR1 concentration corresponding to the threshold sTNFR1 score. Because this score only provided prognostic information in conversion according to CDR-SB but not consensus diagnosis, a predict sTNFR1 score (ysTNFR1) was further calculated using linear recombination of sTNFR1, sTNFR2, and sVCAM-1 concentrations. Values for p-TauTau₁₈₁ and ysTNFR1 were then tested in the Atlanta replication cohort for diagnostic conversion (CDR-SB not available for many participants).

A similar conversion analysis was used to assess biomarker scores' effects on significant decline in AD dementia in the 36 months following CSF collection, with a threshold of CDR-SB >.7.8 (1.5 standard deviation above the mean) derived from the Knight cohort.48 For NC, conversion to MCI or global CDR≥0.5 was examined in the 60 months following CSF collection.

### Example 12: Data Availability

All ADNI data (including CSF inflammatory protein measures) are available for public access at adni.loni.usc.edu contingent on adherence to the ADNI Data Use Agreement, and all data in the validation cohorts are available from RUResearch Data Portal (https ://rucore.libraries. rutgers.edu/research/).

Discussion

Genetic and neuropathologic studies have pointed towards detrimental roles for inflammation in AD, but neuroinflammation can also trigger neuroprotective and pro-survival cascades (Dong, Y. et al. Proceedings of the National Academy of Sciences of the United States of America 113, 12304-12309, doi:10.1073/pnas.1605195113 (2016); Bartsch, J. W. et al. J Neurosci 30, 12210-12218, doi:10.1523/JNEUROSCI.1520-10.2010 (2010).). Four non-sTREM2 products of TACE/ADAM17 were reproducibly identified to co-vary across diagnosis in ADNI independent of core AD biomarkers, and a similar trend was replicated in two separate cohorts for three of these proteins which were measured. Higher levels of a PCA-derived score consisting of sTNFR1, sTNFR2, and sVCAM1 were further found—but not sTREM2—to associate with a halved risk of conversion among MCI participants with predicted ADNC in two independent cohorts. Consistent with the goal of a prognostic biomarker to identify likelihood of a clinical event or progression in patients who have the disease (Group, F.-N. B. W. (eds Food and Drug Administration (US) & National institutes of Health (US)) 23-26 (Food and Drug Administration and National Institutes of Health, Silver Spring (Md.) Bethesda (Md.), 2016)), CSF-based prognostic biomarker can complement core AD diagnostic biomarkers in very early AD.

Commercial assays were used which were independently assessed for intermediate precision (Hu, W. T. et al. Acta Neuropathol 119, 669-678, doi:10.1007/s00401-010-0667-0 (2010)). The large sample size in ADNI and moderate sample sizes in the two Atlanta cohorts allowed detection of extraordinarily consistent PCs (families) across these cohorts, even when biomarkers within the same PC have been reported to derive from different cell types. Co-variance in their levels afforded the opportunity where one member of the family may be the best clinical biomarker while others better informed biological specificity. For example, sTNFR2 was more exclusively released by microglia and monocytes than sTNFR1 (which was released from all cell types), and non-endothelial VCAM1 was also expressed on microglia (Peterson, J. W. et al. Journal of neuropathology and experimental neurology 61, 539-546, doi:10.1093/jnen/61.6.539 (2002)). Therefore, sTNFR2/sVCAM1 may provide more cell-type specificity even though sTNFR1 had greater loading on the PC score.

Conversely, while some TACE/ADAM17 sheddase products were reliably found in the same PC, others — TNFa, sTREM2 — were not. This was not surprising for biological and statistical reasons. Biologically, factors such as circulation, interstitial exchange (Ichimura, T., Fraser, P. A. & Cserr, H. F. Brain Res 545, 103-113, doi:10.1016/0006-8993(91)91275-6 (1991)), bulk flow (Cserr, H. F., Cooper, D. N., Suri, P. K. & Patlak, C. S. Am J Physiol 240, F319-328, doi:10.1152/ajprenal.1981.240.4.F319 (1981); Groothuis, D. R. et al. J Cereb Blood Flow Metab 27, 43-56, doi:10.1038/sj jcbfm.9600315 (2007)), and lymphatic clearance (Louveau, A. et al. Nature 523, 337-341, doi:10.1038/nature14432 (2015)) can all influence measured CSF protein levels beyond surface cleavage. Statistically, PCA exploits underlying proteins levels' data structures to reduce the number of dimensions while maximizing variance explained, but a modest correlation between two variables across hidden clusters may mask the variables' more independent relationships (Lever, J., Krzywinski, M. & Altman, N. Nature Methods 14, 641-642, doi:10.1038/nmeth.4346 (2017)). Associated protein level changes independent of AD status/biomarkers than proteins differed in levels between NC and AD dementia. This data-driven approach therefore provided information not otherwise available through knowledge-based pathway analysis. These loading profiles may be tested using drugs known to alter one or more of the proteins.

In two well-characterized independent cohorts, higher levels of CSF sTNFR1-related proteins (sTNFR1, sTNFR2, sVCAM1) were associated with reduced cognitive decline in those with MCI independent of markers of ADNC. A reproducible scheme to nominate and analyze CSF inflammatory as biomarkers in neurodegeneration was developed, and results of such methods validated prior findings from smaller studies (Taipa, R. et al. Neurobiology of aging 76, 125-132, doi:10.1016/j.neurobiolaging.2018.12.019 (2019); Magalhaes, T. N. C. et al. Molecular neurobiology 55, 5689-5697, doi:10.1007/s12035-017-0795-9 (2018)). The inflammatory alterations identified can reflect microglial phenotype evolution along the AD disease continuum, and autopsy- or PET-based analysis of people in early AD stages. Because commercial assays for sTNFR1, sTNFR2, and sVCAM-1 had high intermediate precision, these biomarkers can be readily introduced into existing workflows to provide prognostic information and improve clinical trial design.

Higher levels of proteins related to soluble tumor necrosis factor receptor 1 were associated with reduced risk of conversion to dementia in the multi-centered (p=0.027) and independent (p=0.038) cohorts of people with mild cognitive impairment due to predicted Alzheimer's disease (p=0.027), while higher soluble TREM2 levels were associated with slower decline in the dementia stage of Alzheimer's disease. These inflammatory proteins, including but not limited to, biomarkers sTNFR1, sTNFR2, sVCAM1, thus provided prognostic information independent of established Alzheimer's markers, such as but not limited to, beta-amyloid 1-42 (Ab42), total tau (t-Tau), and tau phosphorylated at threonine 181 (p-Tau₁₈₁).

TABLES

TABLE 1 illustrates subjects included in a study from ADNI, with p-values shown for all continuous factors without log₁₀ transformation but also for CSF inflammatory proteins after log₁₀ transformation. Note two analytes (*) have concentrations in the range of ng/mL.

TABLE 1

| | | | | | | | | p for |
|---|---|---|---|---|---|---|---|---|
| | NC | | MCI | | AD | | | log₁₀ |
| | (n = 111) | | (n = 174) | | (n = 97) | | P | values |
| Male (%) | 57 | (51%) | 112 | (64%) | 56 | (58%) | 0.090 | |
| Age, mean (SD) | 75.8 | (5.3) | 75.2 | (7.6) | 75.1 | (7.8) | 0.659 | |
| Education, mean (SD) | 15.7 | (2.9) | 15.8 | (2.9) | 15.2 | (3.0) | 0.246 | |
| Race | | | | | | | 0.001 | |
| Asian (%) | 0 | | 5 | (3%) | 0 | | | |
| Black/African American (%) | 9 | (8%) | 3 | (2%) | 0 | | | |
| white (%) | 102 | (92%) | 166 | (95%) | 97 | (100%) | | |
| Non-Hispanic (%) | 109 | (98%) | 170 | (98%) | 96 | (99%) | 0.595 | |
| BMI (kg/m²) | 26.9 | (4.4) | 25.9 | (3.9) | 25.4 | (3.6) | 0.017 | |
| SBP (mmHg) | 133.8 | (16.0) | 132.3 | (15.5) | 134.1 | (17.0) | 0.626 | |
| Having at least one APOE ε4 | 26 | (23%) | 95 | (55%) | 65 | (67%) | <0.001 | |
| allele (%) | | | | | | | | |
| CDR-SB (SD) | 0.05 | (0.24) | 1.69 | (1.00) | 4.48 | (1.88) | <0.001 | |
| CSF biomarkers | | | | | | | | |
| A□42, mean (SD) in pg/mL | 209.8 | (52.7) | 165.6 | (52.4) | 142.5 | (36.7) | <0.001 | |
| t-Tau, mean (SD) in pg/mL | 69.3 | (30.2) | 105.2 | (61.3) | 121.8 | (57.7) | <0.001 | |
| p-Tau₁₈₁, | 27.0 | (17.2) | 37.0 | (21.3) | 43.2 | (19.8) | <0.001 | |
| mean (SD) in pg/mL | | | | | | | | |
| t-Tau/Aβ42 ≥ 0.39 (%) | 32 | (29%) | 120 | (69%) | 85 | (88%) | <0.001 | |
| WU-sTREM2, mean (SD) in | 2427 | (774) | 2366 | (726) | 2474 | (802) | 0.611 | — |
| pg/mL | | | | | | | | |
| MSD-sTREM2, mean (SD) in | 4692 | (2274) | 4529 | (2539) | 4347 | (1975) | 0.628 | 0.871 |
| pg/mL | | | | | | | | |
| MSD-GRN, mean (SD) in | 1598 | (565) | 1612 | (798) | 1626 | (425) | 0.963 | 0.808 |
| pg/mL | | | | | | | | |
| TNF-□, mean (SD) in pg/mL | 1.91 | (1.44) | 1.83 | (1.20) | 1.73 | (0.46) | 0.522 | 0.837 |
| sTNFR1, mean (SD) in pg/mL | 870 | (227) | 904 | (237) | 874 | (249) | 0.434 | 0.353 |
| sTNFR2, mean (SD) in pg/mL | 1060 | (512) | 1093 | (319) | 1048 | (284) | 0.597 | 0.299 |
| TGF□1, mean (SD) in pg/mL | 109 | (42) | 108 | (42) | 107 | (37) | 0.951 | 0.987 |
| TGF□2, mean (SD) in pg/mL | 159 | (39) | 161 | (53) | 159 | (47) | 0.398 | 0.168 |
| TGF□3, mean (SD) in pg/mL | 9.2 | (23.3) | 11.1 | (28.3) | 14.2 | (31.1) | 0.438 | 0.675 |
| IP-10, mean (SD) in ng/mL* | 5.47 | (1.78) | 5.08 | (2.06) | 5.01 | (1.95) | 0.253 | 0.117 |
| IL-6, mean (SD) in pg/mL | 4.78 | (3.33) | 5.27 | (5.78) | 5.03 | (4.74) | 0.676 | 0.970 |
| IL-7, mean (SD) in pg/mL | 1.49 | (2.75) | 1.16 | (0.79) | 1.41 | (1.26) | 0.238 | 0.338 |
| IL-9, mean (SD) in pg/mL | 3.70 | (1.91) | 3.33 | (1.46) | 3.45 | (1.65) | 0.239 | 0.416 |
| IL-10, mean (SD) in pg/mL | 5.80 | (2.74) | 7.97 | (28.55) | 5.57 | (2.60) | 0.532 | 0.431 |
| IL-12p40, mean (SD) in pg/mL | 1.11 | (1.03) | 5.81 | (48.01) | 1.17 | (1.03) | 0.470 | 0.159 |
| IL-21, mean (SD) in pg/mL | 12.93 | (14.71) | 11.78 | (12.10) | 12.10 | (12.46) | 0.766 | 0.820 |
| sICAM-1, mean (SD) in pg/mL | 355.4 | (184.1) | 400.2 | (215.6) | 368.7 | (187.3) | 0.154 | 0.100 |
| sVCAM-1, mean (SD) in | 41.3 | (2.10) | 44.7 | (26.1) | 48.7 | (67.4) | 0.413 | 0.468 |
| ng/mL* | | | | | | | | |

TABLE 2 presents PCA of CSF AD and inflammatory proteins in ADNI and two replication Cohorts B and C, with loading values of proteins consistently associated with two or more PCs shown. *Proteins found in the same PC across ADNI diagnostic groups. Levels of IL-6, IL-12—p40, IL-21, progranulin, and TGFβ1/2/3 were not measured in either replication cohort, and IP-10 was not measured in Cohort C. †Different sTREM2 assays used between ADNI and the replication cohorts.

TABLE 2

| | | ADNI | | | Cohort B | Cohort C |
|---|---|---|---|---|---|---|
| | | NC | MCI | AD | NC, MCI, AD | NC |
| | | (n = 85) | (n = 129) | (n = 68) | (n = 68) | (n = 47) |
| PC1 | t-Tau* | 0.724 | 0.871 | 0.831 | 0.577 | 0.734 |
| | p-Tau₁₈₁* | 0.805 | 0.866 | 0.826 | 0.474 | 0.727 |
| | Aβ42* | −0.752 | −0.745 | −0.612 | −0.781 | −0.595 |
| | t-Tau/Aβ42* | 0.710 | 0.675 | 0.774 | 0.923 | 0.925 |
| PC2 | sTNFR1* | 0.869 | 0.903 | 0.884 | 0.895 | 0.851 |
| | STNRF2* | 0.784 | 0.883 | 0.881 | 0.859 | 0.850 |
| | sVCAM1* | 0.833 | 0.869 | 0.813 | 0.833 | 0.825 |
| | TGFβ1* | 0.533 | 0.469 | 0.561 | N.D. | N.D. |
| | sICAM1* | 0.502 | 0.467 | 0.661 | | |
| | IP-10 | | | 0.484 | 0.602 | N.D. |
| PC3 | MSD-sTREM2* | 0.876 | 0.883 | 0.876 | 0.639† | 0.787† |
| | WU-sTREM2* | 0.830 | 0.882 | 0.819 | | |
| | Progranulin | 0.519 | | 0.640 | N.D. | N.D. |
| PC4 | IL-6* | 0.811 | 0.799 | 0.828 | N.D. | N.D. |
| | IL-10* | 0.691 | 0.662 | 0.713 | 0.922 | 0.812 |
| PC5 | TGFβ2* | 0.885 | 0.820 | 0.754 | N.D. | N.D. |
| | TGFβ1* | 0.598 | 0.716 | 0.752 | N.D. | N.D. |
| | TGFβ3 | | 0.538 | 0.705 | N.D. | N.D. |
| PC6 | IL-7* | 0.866 | 0.810 | 0.797 | 0.912 | 0.942 |
| | TNF-α* | 0.753 | 0.429 | 0.707 | 0.757 | |
| | IL-9 | | 0.601 | 0.647 | | |
| PC7 | IP-10 | 0.722 | 0.705 | | | N.D. |
| | IL12-p40 | 0.686 | 0.772 | | N.D. | N.D. |
| | IL-9 | 0.571 | | | 0.865 | |
| PC8 | IL-21 | 0.458 | 0.904 | | N.D. | N.D. |
| | sICAM1 | | −0.624 | | 0.934 | |

TABLE 3 shows a linear mixed modeling of longitudinal CDR-SB changes according to basic baseline factors across the entire cohort. Significant interaction terms between Time (measured in months) and baseline factors were interpreted as baseline factors' effects on the CDR-SB slope of change over time.

TABLE 3

| | |
|---|---|
| 8 (95% confidence | |
| interval) | P |

Baseline diagnosis

| | |
|---|---|
| NC | Reference |

| | | | |
|---|---|---|---|
| MCI | 1.552 | (1.223, 1.860) | <0.001 |
| AD dementia | 4.348 | (3.938, 4.758) | <0.001 |
| Baseline diagnosis X Months | | | |

| | |
|---|---|
| NC | Reference |

| | | | |
|---|---|---|---|
| MCI | 0.030 | (0.001, 0.059) | 0.043 |
| AD dementia | 0.076 | (0.016, 0.137) | 0.013 |
| Age | 0.002 | (−0.016, 0.021) | 0.793 |
| Age X Months | 0.002 | (0.002, 0.003) | <0.001 |
| Female sex | 0.096 | (−0.372, 8.180) | 0.495 |
| Female sex X Months | 0.015 | (−0.003, 0.034) | 0.104 |
| Education | 0.032 | (−0.013, 0.077) | 0.166 |
| Education X Months | 0.002 | (−0.001, 0.005) | 0.201 |
| Predicted ADNC | 0.046 | (−0.274, 0.367) | 0.775 |
| Predicted ADNC X Months | 0.020 | (−0.012, 0052) | 0.216 |
| Predicted ADNC X | | | |
| Diagnosis X Months | | | |

| | |
|---|---|
| NC | Reference |

| | | | |
|---|---|---|---|
| MCI | 0.048 | (0.007, 0.089) | 0.020 |
| AD dementia | 0.070 | (0.001, 0.138) | 0.045 |

TABLE 4 presents the mean and standard deviation values for log₁₀-transformed cytokine values from ADNI NC participants.

TABLE 4

| | Mean ± SD |
|---|---|
| log₁₀(t-Tau) | 1.808 ± 0.172 |
| log₁₀(p-Tau₁₈₁) | 1.344 ± 0.202 |
| log₁₀(MSD-sTREM2 MSD) | 3.556 ± 0.238 |
| log₁₀(WU-sTREM2) | 2451 ± 762 |
| log₁₀(MSD-GRN) | 3.180 ± 0.108 |
| log₁₀(TNFα) | 0.233 ± 0.173 |
| log₁₀(sTNFR1) | 2.924 ± 0.116 |
| log₁₀(sTNFR2 | 2.999 ± 0.132 |
| log₁₀(sVCAM1) | 4.569 ± 0.199 |
| log₁₀(sICAM1) | 2.503 ± 0.206 |
| log₁₀(IL-6) | 0.513 ± 0.224 |
| log₁₀(IL-7) | −0.068 ± 0.432 |
| log₁₀(IL-9) | 0.513 ± 0.224 |
| log₁₀(IL-10) | 0.722 ± 0.187 |
| log₁₀(IL-12-p40) | −0.480 ± 1.006 |
| log₁₀(IL-21) | 0.737 ± 0.712 |
| log₁₀(IP-10) | 3.712 ± 0.151 |
| log₁₀(TGFβ1) | 2.005 ± 0.171 |
| log₁₀(TGFβ2) | 2.188 ± 0.110 |
| log₁₀(TGFβ3) | 0.555 ± 0.403 |

TABLE 5 illustrates the relationship between CSF cytokine levels, clinical diagnosis, and CSF biomarker profile using ANCOVA adjusting for age, sex, and APOE ε4 status (F- and p-values are shown; p<0.01 used as threshold to adjust for multiple comparisons). Among the 15 CSF inflammatory proteins analyzed in this cohort, CSF levels of four analytes—sTNF-R1, sTNF-R2, TGF-β1, and sICAM1—differed according to predicted ADNC status (p<0.001 for all), and TGF-β2 levels differed according to baseline diagnosis (p=0.006). As expected for the core CSF AD biomarkers, Aβ42 levels differed according to baseline diagnosis (p=0.002), and levels of all three markers (Aβ42, t-Tau, p-Tau₁₈₁, p<0.001) differed according to predicted ADNC status. Neither sTREM2 or progranulin levels varied according to baseline diagnosis or predicted ADNC status in this cohort.

TABLE 5

| | Diagnosis | t-Tau/Aβ4220.39 | Diagnosis X t-Tau/Aβ4220.39 |
|---|---|---|---|
| TNF-α | 1.153, 0.317 | 1.598, 0.207 | 0.119, 0.888 |
| sTNFR1 | 2.248, 0.107 | 17.473, <0.001 | 1.927, 0.147 |
| sTNFR2 | 2.016, 0.135 | 22.298, <0.001 | 0.670, 0.513 |
| IL-6 | 1.597, 0.204 | 0.003, 0.956 | 3.031, 0.050 |
| IL-7 | 0.152, 0.859 | 2.995, 0.084 | 0.661, 0.517 |
| IL-12p40 | 1.486, 0.228 | 0.314, 0.576 | 0.438, 0.646 |
| IP-10 | 0.612, 0.543 | 3.747, 0.054 | 0.364, 0.695 |
| IL-10 | 0.177, 0.838 | 0.071, 0.789 | 0.081, 0.923 |
| IL-9 | 2.574, 0.078 | 6.647, 0.010 | 0.155, 0.856 |
| IL-21 | 0.947, 0.389 | 3.892, 0.049 | 1.336, 0.264 |
| TGFβ1 | 2.460, 0.087 | 12.845, <0.001 | 1.685, 0.187 |
| TGFβ2 | 5.112, 0.006 | 3.944, 0.048 | 2.737, 0.066 |
| TGFβ3 | 0.993, 0.372 | 2.896, 0.090 | 3.031, 0.049 |
| sICAM1 | 1.313, 0.270 | 14.029, <0.001 | 0.242, 0.785 |
| sVCAM1 | 0.024, 0.976 | 4.068, 0.044 | 2.543, 0.080 |
| MSD-sTREM2 | 0.188, 0.829 | 1.082, 0.299 | 0.485, 0.616 |
| WU-sTREM2 | 0.355, 0.701 | 2.532, 0.113 | 0.247, 0.782 |
| progranulin | 0.173, 0.841 | 0.277, 0.599 | 0.314, 0.730 |
| Aβ42 | 6.321, 0.002 | 196.192, <0.001 | 2.414, 0.091 |
| t-Tau | 3.058, 0.048 | 208.161, <0.001 | 3.212, 0.041 |
| p-Tau₁₈₁ | 1.411, 0.245 | 160.684, <0.001 | 3.024, 0.050 |

TABLE 6 presents PCA of MCI participants from ADNI, showing loading (≥0.100) with missing values excluded (loading with missing values replaced with means in parentheses). Variables with loading value ≤0.400 in both models are shown in bold).

TABLE 6

| | PC1 | | PC2 | | PC3 | | PC4 | |
|---|---|---|---|---|---|---|---|---|
| t-Tau | 0.871 | (0.871) | 0.356 | (0.331) | | | | |
| p-Tau₁₈₁ | 0.866 | (0.906) | 0.128 | (0.102) | | | | |
| Aβ42 | −0.745 | (−0.792) | 0.343 | (0.34 [symbol] ) | −0.142 | | −0.141 | |
| t-Tau/Aβ42 | 0.675 | (0.322) | 0.244 | (0.161) | | | | |
| sTNFR1 | 0.137 | (0.173) | 0.903 | (0.848) | 0.281 | (0.297) | | |
| sTNFR2 | 0.255 | (0.223) | 0.883 | (0.785) | 0.343 | (0.324) | (0.219) | |
| sVCAM1 | | | 0.869 | (0.836) | 0.202 | (0.168) | 0.165 | (0.135) |
| sICAM1 | 0.333 | (0.292) | 0.467 | (0.458) | | | (0.150) | |
| IP-10 | −0.136 | (−0.102) | 0.186 | (0.224) | 0.255 | (0.268) | | |
| MSD- | | | 0.471 | (0.383) | 0.883 | (0.789) | (0.135) | |
| sTREM2 | | | | | | | | |
| WU- | | | 0.263 | (0.215) | 0.882 | (0.850) | | |
| sTREM2 | | | | | | | | |
| progranulin | | | 0.588 | (0.325) | | | 0.530 | (0.662) |
| IL-6 | | | | | | | 0.799 | (0.824) |
| IL-10 | −0.118 | (−0.119) | 0.155 | (0.125) | 0.212 | (0.150) | 0.662 | (0.725) |
| TGFβ2 | | | (−0.188) | | | | | |
| TGFβ1 | 0.137 | | 0.469 | (0.510) | | | | |
| TGFβ3 | 0.176 | | | | −0.128 | (−0.128) | | |
| IL-7 | | | −0.116 | | | | 0.2 [symbol] 4 | (0.283) |
| TNF-α | | | 0.378 | (0.331) | | | 0.157 | (0.506) |
| IL-9 | (0.100) | | 0.288 | (0.230) | (0.143) | | (−0.195) | |
| IL12-p40 | 0.123 | | | | | | (0.141) | |
| IL-21 | | | 0.153 | | (−0.119) | | | |
| | PC5 | | PC6 | | PC7 | | PC8 | |
| t-Tau | | | | | | | | |
| p-Tau₁₈₁ | | | | | | | | |
| Aβ42 | | | | | | | 0.118 | |
| t-Tau/Aβ42 | 0.123 | | | | | | | |
| sTNFR1 | | | 0.144 | (0.127) | 0.124 | (0.122) | | |
| sTNFR2 | | | (0.107) | | 0.226 | (0.167) | | |
| sVCAM1 | | | | | 0.161 | | 0.124 | (0.166) |
| sICAM1 | −0.198 | | 0.378 | (0.211) | 0.199 | (0.126) | −0.624 | (−0.581) |
| IP-10 | −0.153 | (−0.120) | (0.119) | | 0.705 | (0.672) | 0.100 | (0.139) |
| MSD- | | | 0.199 | (0.132) | 0.151 | | | |
| sTREM2 | | | | | | | | |
| WU- | | | | | 0.212 | (0.170) | −0.142 | |
| sTREM2 | | | | | | | | |
| progranulin | | | (−0.206) | | (0.126) | | (0.174) | |
| IL-6 | | | 0.109 | (0.155) | (−0.118) | | (−0.107) | |
| IL-10 | −0.158 | | | | 0.459 | (0.459) | | |
| TGFβ2 | 0.820 | (0.903) | | | −0.150 | (−0.124) | | |
| TGFβ1 | 0.354 | (0.694) | 0.270 | | (0.134) | | −0.112 | (−159) |
| TGFβ3 | 0.538 | (0.612) | | | | | | |
| IL-7 | −0.103 | | 0.810 | (0.769) | 0.245 | | 0.393 | (0.244) |
| TNF-α | | | 0.429 | (0.435) | 0.618 | (0.382) | | |
| IL-9 | | | 0.601 | (0.739) | 0.155 | (0.199) | −0.105 | (−0.120) |
| IL12-p40 | | | 0.223 | (0.112) | 0.772 | (0.849) | | |

| | | | |
|---|---|---|---|
| IL-21 | (0.106) | 0.904 | (0.924) |

[symbol]

indicates data missing or illegible when filed

TABLE 7 presents MCI models of longitudinal ADNI-Mem-EF changes using biomarker family scores (from PCA), 0-60 months after CSF collection (ΔAIC=11.7, significant factors highlighted in italics (Baseline Cognitive Z; Age X Months; AD score X Months; sTNFR1 score X Months; Female sex X months, AIC=386.4; AD score, AIC=386.4) with p<0.00625 used for CSF biomarkers to adjust for multiple comparisons).

TABLE 7

| | AIC = 386.4 | | | AIC = 374.7 | | |
|---|---|---|---|---|---|---|
| | B (95% CI) | | P | B (95% CI) | | P |
| Months | 0.013 | (−0.012, 0.038) | 0.298 | 0.028 | (0.002, 0.054) | 0.033 |
| Baseline Cognitive Z | 0.942 | (0.890, 0.995) | <0.001 | 0.943 | (0.911, 0.996) | <0.001 |
| Baseline Cognitive Z X Months | 0.0054 | (0.0006, 0.0101) | 0.027 | 0.0004 | (0, 0.009) | 0.070 |
| Female sex | 0.011 | (−0.053, 0.075) | 0.734 | 0.013 | (−0.052, 0.078) | 0.695 |
| Femail sex X Months | −0.008 | (−0.014, −0.003) | 0.004 | −0.006 | (−0.012, 0.001) | 0.019 |
| Age | 0 | (−0.004, 0.004) | 0.898 | 0 | (−0.004, 0.005) | 0.871 |
| Age X Months | −0.0004 | (−0.0008, 0.0001) | 0.006 | −0.0006 | (−0.0009, −0.0003) | <0.001 |
| APOE e4+ | 0.075 | (0.010, 0.140) | 0.023 | 0.057 | (0, 0.116) | 0.060 |
| AD score | −0.048 | (−0.081, −0.015) | 0.005 | −0.046 | (−0.080, −0.011) | 0.009 |
| AD score X Months | −0.006 | (−0.009, −0.003) | <0.001 | −0.008 | (−0.011, −0.005) | <0.001 |
| sTNFR1 score | | | | −0.010 | (−0.042, 0.022) | 0.547 |
| sTNFR1 score X Months | | | | 0.005 | (0.002, 0.008) | <0.001 |
| AD score X sTNFR1 score | | | | 0.007 | (−0.026, 0.040) | 0.667 |
| AD score X sTNFR1 score X Months | | | | 0.002 | (0, 0.005) | 0.078 |

TABLE 8 provides MCI models of longitudinal CDR-SB changes using biomarker scores (from PCA), 0-60 months after CSF collection (ΔAIC=8.2 for biomarker family scores when sTNFR1 score was introduced). Using p-Tau₁₈₁ and sTNFR1 levels showed similar results (ΔAIC=16.5). Significant factors are highlighted in italics (Baseline CDR-SB; Age X Months; Core AD score X Months; sTNFR1 score X Months; Zlog₁₀(sTNFR1) X Months; Months, AIC=2834.1) at p<0.00625 for CSF biomarker PC score/biomarkers to adjust for multiple comparisons).

TABLE 8

| | AIC = 2842.3 | | | AIC = 2834.1 | | |
|---|---|---|---|---|---|---|
| | B (95% CI) | | P | B (95% CI) | | P |
| Months | −0.094 | (−0.203, 0.014) | 0.090 | −0.158 | (−0.272, −0.045) | 0.006 |
| Baseline CDR-SB | 0.912 | (0.804, 1.019) | <0.001 | 0.906 | (0.799, 1.013) | <0.001 |
| Baseline CDR-SB X Months | 0.013 | (0.001, 0.026) | 0.032 | 0.012 | (0, 0.024) | 0.059 |
| Age | 0.005 | (−0.008, 0.018) | 0.466 | 0.007 | (−0.007, 0.022) | 0.339 |
| Age X Months | 0.002 | (0.001, 0.003) | 0.003 | −0.003 | (0.001, 0.004) | <0.001 |
| Core AD score | −0.061 | (−0.164, 0.041) | 0.241 | −0.060 | (−0.163, 0.042) | 0.246 |
| Core AD score X Months | 0.028 | (0.016, 0.040) | <0.001 | 0.029 | (0.017, 0.040) | <0.001 |
| sTNFR1 score | | | | −0.026 | (−0.139, 0.086) | 0.642 |
| sTNFR1 score X Months | | | | 0.020 | (−0.033, −0.008) | 0.002 |

| | AIC = 2836.2 | | | AIC = 2819.7 | | |
|---|---|---|---|---|---|---|
| | B (95% CI) | | P | B (95% CI) | | P |
| Months | −0.126 | (−0.238, −0.014) | 0.028 | −0.187 | (−0.306, −0.068) | 0.002 |
| Baseline CDR-SB | 0.903 | (0.795, 1.010) | <0.001 | 0.905 | (0.793, 1.016) | <0.001 |
| Baseline CDR-SB X Months | 0.017 | (0.004, 0.030) | 0.008 | 0.016 | (0.004, 0.029) | 0.010 |
| Age | 0.004 | (−0.010, 0.018) | 0.579 | 0.004 | (−0.012, 0.020) | 0.626 |
| Age X Months | 0.002 | (0.001, 0.004) | 0.002 | 0.003 | (0.001, 0.004) | <0.001 |
| APOE ε4+ | −0.107 | (−0.329, 0.115) | 0.345 | −0.106 | (−0.336, 0.123) | 0.363 |
| APOE ε4+ X Months | 0.032 | (0.007, 0.058) | 0.012 | 0.032 | (0.007, 0.057) | 0.011 |
| Zlog₁₀(p-Tau₁₈₁) | −0.050 | (−0.154, 0.054) | 0.345 | −0.047 | (−0.158, 0.065) | 0.411 |
| Zlog₁₀(p-Tau₁₈₁) X Months | 0.009 | (0.001, 0.017) | 0.026 | 0.011 | (0.003, 0.019) | 0.007 |
| Zlog₁₀(s-TNFR1) | | | | 0.014 | (−0.115, 0.144) | 0.825 |
| Zlog₁₀(s-TNFR1) X Months | | | | −0.021 | (−0.034, −0.007) | 0.004 |

TABLE 9 presents factors associated with rates of cognitive decline in ADNI MCI participants with ADNI-Mem-EF or CDR-SB as outcome, 0-60 months after CSF collection (see TABLE 7 and TABLE 12 for comparison between models without and with sTNDF1 scores, with improvement in Akaike Information Criterion (AIC) of 11.7 and 8.2; significant factors highlighted in bold with p<0.00625 used for CSF biomarkers to adjust for multiple comparisons).

TABLE 9

| | ADNI-Mem-EF | | | CDR-SB | | |
|---|---|---|---|---|---|---|
| Cognitive measure | B (95% CI) | | P | B (95% CI) | | P |
| Months | 0.028 | (0.002, 0.054) | 0.033 | −0.158 | (−0.272, −0.045) | 0.006 |
| Baseline Cognitive | 0.943 | (0.911, 0.996) | <0.001 | 0.906 | (0.799, 1.013) | <0.001 |
| measure | | | | | | |
| Baseline Cognitive | 0.004 | (0, 0.009) | 0.070 | 0.012 | (0, 0.024) | 0.059 |
| measure X Months | | | | | | |
| Female sex | 0.013 | (−0.052, 0.078) | 0.695 | | | |
| Female sex X Months | −0.006 | (−0.012, 0.001) | 0.019 | | | |
| Age | 0 | (−0.004, 0.005) | 0.871 | 0.007 | (−0.007, 0.022) | 0.339 |
| Age X Months | −0.0006 | (−0.0009, −0.0003) | <0.001 | 0.003 | (0.001, 0.004) | <0.001 |
| AD score | −0.046 | (−0.080, −0.011) | 0.009 | −0.060 | (−0.163, 0.042) | 0.246 |
| AD score X Months | −0.008 | (−0.011, −0.005) | <0.001 | 0.029 | (0.017, 0.040) | <0.001 |
| sTNFR1 score | −0.010 | (−0.042, 0.022) | 0.547 | −0.026 | (−0.139, 0.086) | 0.642 |
| sTNFR1 score X Months | 0.005 | (0.002, 0.008) | <0.001 | −0.020 | (−0.033, −0.008) | 0.002 |
| AD score X sTNFR1 score | 0.007 | (−0.026, 0.040) | 0.667 | | | |
| AD score X sTNFR1 score | 0.002 | (0, 0.005) | 0.078 | | | |
| X Months | | | | | | |

TABLE 10 presents dementia models of longitudinal cognitive decline using biomarker scores, with average ADNI-Mem-EF or CDR-SB as outcome, 0-36 months (ΔAIC=9.56 and 8.20; significant factors highlighted in italics (Months, AIC=92.29, AIC=82.73; Baseline ADNI-Mem-EF; Baseline CDR-SB; Baseline CDR-SB X Months; sTREM2 score X Months, AIC=1172.8) with p<0.00625 used for CSF biomarkers to adjust for multiple comparisons).

TABLE 10

| | AIC = 92.29 | | | AIC = 82.73 | | |
|---|---|---|---|---|---|---|
| | B (95% CI) | | P | B (95% CI) | | P |
| Months | −0.086 | (−0.137, −0.036) | 0.001 | −0.070 | (−0.121, −0.020) | 0.007 |
| Baseline ADNI-Mem-EF | 0.999 | (0.947, 1.051) | <0.001 | 0.997 | (0.946, 1.047) | <0.001 |
| Age | 0.001 | (−0.003, 0.006) | 0.542 | 0.001 | (−0.004, 0.005) | 0.760 |
| Age X Months | 0.0008 | (0.0002, 0.015) | 0.015 | 0.0006 | (0, 0.0012) | 0.070 |
| APOE ε4+ | 0.079 | (0.010, 0149) | 0.032 | 0.072 | (0.004, 0.140) | 0.038 |
| Core AD biomarker score | −0.003 | (−0.040, 0.033) | 0.852 | −0.004 | (−0.040, 0.032) | 0.832 |
| Core AD biomarker score X | −0.003 | (0.008, 0.002) | 0.177 | −0.004 | (−0.009, 0.001) | 0.115 |
| Months | | | | | | |
| sTREM2 score | | | | 0.027 | (−0.008, 0.062) | 0.127 |
| sTREM2 score X Months | | | | 0.006 | (0.001, 0.011) | 0.015 |

| | AIC = 1181.0 | | | AIC = 1172.8 | | |
|---|---|---|---|---|---|---|
| | B (95% CI) | | P | B (95% CI) | | P |
| Months | 0.034 | (−0.042, 0.110) | 0.375 | 0.040 | (−0.033, 0.111) | 0.283 |
| Baseline CDR-SB | 0.968 | (0.875, 1.060) | <0.001 | 0.968 | (0.876, 1.061) | <0.001 |
| Baseline CDR-SB X Months | 0.028 | (0.012, 0.045) | 0.001 | 0.028 | (0.012, 0.043) | 0.001 |
| Core AD biomarker score | −0.087 | (−0.256, 0.081) | 0.306 | −0.091 | (−0.259, 0.077) | 0.285 |
| Core AD biomarker score X | 0.024 | ( )−0.002, 0.050) | 0.070 | 0.024 | (0, 0.005) | 0.050 |
| Months | | | | | | |
| sTREM2 score | | | | 0.004 | (−0.162, 0.170) | 0.964 |
| sTREM2 score X Months | | | | −0.040 | (−0.065, −0.016) | 0.001 |

TABLE 11 illustrates an NC model—ADNI-Mem-EF as outcome, 0-60 months (ΔAIC=6.0, significant factors highlighted in italics (Baseline ADNI-Mem-EF; Baseline ADNI-Mem-EF X Month; Baseline ADNI-Mem-EF X Month²; Core AD score x Month; IL6 score X Month; i16 score X Month²; Month, AIC=223.35; Month², AIC=223.35; Age X Month, AIC=223.35) with p<0.00625 used for CSF biomarkers to adjust for multiple comparisons). ²Irwin et al. Arch Neurol. 69:1018-1025, 2012.

TABLE 11

| | AIC = 229.36 | | | AIC = 223.35 | | |
|---|---|---|---|---|---|---|
| | B (95% CI) | | P | B (95% CI) | | P |
| Month | 0.076 | (0.017, 0.134) | 0.011 | 0.102 | (0.046, 0.158) | <0.001 |
| Month² | −0.001 | (−0.002, 0) | 0.025 | −0.0016 | (−0.0026, −0.0007) | <0.001 |
| Baseline ADNI-Mem-EF | 0.983 | (0.889, 1,077) | <0.001 | 0.982 | (0.889, 1.075) | <0.001 |
| Baseline | −0.018 | (−0.026, −0.010) | <0.001 | −0.019 | (−0.027, −0.011) | <0.001 |
| ADNI-Mem-EFX | | | | | | |
| Month | | | | | | |
| Baseline | 0.0004 | (0.0002, 0.0005) | <0.001 | 0.0004 | (0.0002, 0.0005) | <0.001 |
| ADNI-Mem-EFX | | | | | | |
| Month² | | | | | | |
| Age | −0.003 | (−0.011, 0.005) | 0.465 | −0.002 | (−0.011, 0.006) | 0.541 |
| Age X Month | −0.008 | (−0.014, 0) | 0.036 | −0.001 | (−0.002, 0) | 0.004 |
| Age X Month² | 1.0E−5 | (−1.9E−6, 2.2E−5) | 0.098 | 1.5E−5 | (3.4E−6, 2.7E−5) | 0.012 |
| Male sex | −0.008 | (−0.097, 0.081) | 0.859 | | | |
| Male sex X Months | 0.007 | (−0.001, 0.014) | 0.093 | | | |
| Male sex X Months² | −0.0001 | (−0.0002, 0) | 0.065 | | | |
| Core AD score | 0.007 | (−0.032, 0.046) | 0.737 | 0.004 | (−0.35, 0.044) | 0.825 |
| Core AD score x Month | −0.003 | (−0.005, −0.002) | 0.001 | −0.003 | (−0.005, −0.001) | 0.001 |
| IL6 score | | | | −0.013 | (−0.058, 0.030) | 0.546 |
| IL6 score X Month | | | | 0.005 | (0.002, 0.009) | 0.005 |
| IL6 score X Month² | | | | −8.5E−5 | (−1.4E−4, −3E−5) | 0.004 |

TABLE 12 shows demographic, clinical, and biomarker information for Cohorts B & C. * AD biomarkers were measured using Luminex (Alzbio3, Fujirebio Diagnostics, Malvern, PA) in Cohort B, and ELISA (InnoTest Fujirebio Diagnostics) in Cohort C. The absolute values were well-characterized to result from differences in antibody pairing and assay platforms, and ELISA measures were converted to equivalent Luminex measures using a validated conversion formula (Irwin, D. J. et al. Arch Neurol 69, 1018-1025, doi:10.1001/archneurol.2012.26 (2012)). †A threshold of t-Tau/Aβ42>0.39 for Luminex-derived measures based on a previous autopsy-derived series (Shaw, L. M. et al. Ann Neurol 65, 403-413, doi:10.1002/ana.21610 (2009)) was selected.

TABLE 12

| | Cohorts | | Cohort C | |
|---|---|---|---|---|
| | (n = 126) | | in = (n = 68) | |
| Male (%) | 56 | (44%) | 24 | (35%) |
| Age, mean (SD) | 70.0 | (7.6) | 58.9 | (6.8) |
| Education, mean (SD) | 15.7 | (2.9) | N.A. | |
| Race | | | | |
| Asian (%) | 0 | | 0 | |
| Black/African American (%) | 58 | (46%) | 21 | (31%) |
| Non-Hispanic White (%) | 68 | (54%) | 47 | (69%) |
| Non-Hispanic (%) | 126 | (100%) | 68 | (100%) |
| Having at least one | 64/124 | (51%) | 33 | (48%) |
| APOE ε4 allele (%) | | | | |
| Diagnosis | | | | |
| NC | 51 | (40%) | 68 | (100%) |
| MCI | 50 | (40%) | 0 | |
| AD dementia | 25 | (20%) | 0 | |
| CSF biomarkers | | | | |
| Aβ42, mean (SD) in pg/mL | 210.3 | (133.8) | 709.4 | (186.7)* |
| t-Tau, mean (SD) in pg/mL | 60.2 | (42.6) | 295.9 | (161.9)* |
| p-Tau₁₈₁, | 22.1 | (11.8) | 48.4 | (20.5)* |
| mean (SD) in pg/mL | | | | |
| t-Tau/Aβ42 consist | 47 | (37%) | 8 | (12%)* |
| with AD† (%) | | | | |
| sTREM2, mean (SD) in pg/mL | 339.1 | (115.6) | 340.2 | (116.2) |
| THF-α, mean (SD) in pg/mL | 2.13 | (0.78) | 1.17 | (0.85) |
| sTNFR1, mean (SD) in pg/mL | 622 | (184) | 570 | (162) |
| sTNFR2, mean (SD) in pg/mL | 878 | (308) | 706 | (222) |
| TGFβ1, mean (SD) in pg/mL | N.D. | | N.D. | |
| TGFβ2, mean (SD) in pg/mL | N.D. | | N.D. | |
| TGFβ3, mean (SD) in pg/mL | N.D. | | N.D. | |
| IP-10, mean (SD) in ng/mL | 3.61 | (1.89) | N.D. | |
| IL-6, mean (SD) in pg/mL | N.D. | | N.D. | |
| IL-7, mean (SD) in pg/mL | 3.54 | (2.13) | 1.65 | (0.79) |
| IL-9, mean (SD) in pg/mL | 2.78 | (1.97) | 3.66 | (2.06) |
| IL-10, mean (SD) in pg/mL | 7.25 | (3.60) | 5.74 | (2.52) |
| IL-12p40, mean (SD) in pg/mL | N.D. | | N.D. | |
| IL-21, mean (SD) in pg/mL | N.D. | | N.D. | |
| sICAM-1, mean (SD) in pg/mL | 139.3 | (88.0) | 299.0 | (160.0) |
| sVCAM-1, mean (SD) in ng/mL | 18.2 | (9.9) | 24.9 | (10.7) |

TABLE 13 provides MCI participants in replication cohort (n=49). *Log₁₀-transformed values were analyzed.

TABLE 13

| | Replication MCI cohort | | p (vs. ADNI MCI) |
|---|---|---|---|
| Male (%) | 26 | (65%) | 0.183 |
| Age, mean (SD) | 69.3 | (7.9) | <0.0001 |
| Education, mean (SD) | 16.5 | (2.5) | 0.126 |
| Race | | | |
| Asian (%) | 0 | | |
| Black/African American (%) | 14 | (29%) | |
| white (%) | 35 | (71%) | <0.0001 |
| Non-Hispanic (%) | 49 | (100%) | 0.578 |
| Having at least one APOE ε4 allele | 14/28 | (50%) | 0.684 |
| (%) | | | |
| CSF biomarkers | | | |
| Aβ42, mean (SD) in pg/mL | 179.7 | (142.9) | 0.451 |
| t-Tau, mean (SD) in pg/mL | 62.5 | (36.6) | <0.0001* |
| p-Tau₁₈₁, mean (SD) in pg/mL | 29.6 | (17.3) | 0.0001* |
| t-Tau/Aβ42 ≥ 0.39 (%) | 27 | (55%) | 0.088 |
| sTNFR1 | 645.5 | (179.4) | <0.0001* |

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications and variances which fall within the scope of the appended claims.

All patents and publications mentioned in this specification are herein incorporated by reference in their entirety for their teachings related to the methods and/or products disclosed to the same extent as if each independent patent and publication were specifically and individually indicated to be incorporated by reference.

1. A method of treatment, comprising:
(a) detecting in a cerebral spinal fluid (CSF) sample from a subject, a level of at least two biomarkers selected from the group consisting of: sTNFR1, sTNFR2, and sVCAM1; and
(b) determining progression of Alzheimer's disease (AD) in the subject; and
(c) administering a treatment to the subject based on the progression of AD in the subject.

2. The method of claim 1, wherein the determining of (b) comprises:
generating a biomarker score based on the level of the at least two biomarkers,
wherein the biomarker score of 0 or greater in the CSF sample of the subject indicates a slow progression of Alzheimer's disease (AD),
wherein slow progression is conversion to dementia at a median of 36 months;
wherein the biomarker score of less than 0 in the CSF sample of the subject indicates a fast progression of AD,
wherein fast progression is conversion to dementia at a median of 12-18 months;
thereby determining progression of Alzheimer's disease in the subject.

3. The method of claim 1, further comprising at least one biomarker selected from the group consisting of: beta-amyloid 1-42 (Ab42); total tau (t-Tau); tau phosphorylated at threonine 181 (p-Tau₁₈₁); Transforming Growth Factor 1, 2, and 3 (TGFβ1, TGFβ2, TGFβ3); soluble Intercellular Adhesion Molecule 1 (sICAM1); Tumor Necrosis Factor α (TNFα); Interleukin 6 (IL)-6; IL-7; IL-9; IL-10; IL-12p40; IL-21; and Interferon Gamma-Induced Protein 10 (IP-10), or combinations thereof.

4. The method of claim 2, comprising three biomarkers.

5. The method of claim 3, wherein the biomarker score is: 0.334+0.633×zlog₁₀[biomarker 1]+0.544×zlog₁₀[biomarker 2]−0.230×zlog₁₀[biomarker 3].

6. The method of claim 4, wherein biomarker 1 is sTNFR1; biomarker 2 is sVCAM1; and biomarker 3 is sTNFR2.

7. The method of claim 1, wherein the determining of (b) comprises:
generating a biomarker score based on the level of the at least two biomarkers,
wherein the biomarker score of −0.435 or greater in the CSF sample of the subject indicates a slow progression of Alzheimer's disease (AD),
wherein slow progression is conversion to dementia at a median of 36 months;
wherein the biomarker score of less than -0.435 in the CSF sample of the subject indicates a fast progression of AD,
wherein fast progression is conversion to dementia at a median of 12-18 months;
thereby determining progression of Alzheimer's disease in the subject.

8. The method of claim 6, comprising three biomarkers.

9. The method of claim 7, wherein the biomarker score is: 0.101+0.633×zlog₁₀[biomarker 1]+0.544×zlog₁₀[biomarker 2]−0.230×zlog₁₀[biomarker 3].

10. The method of claim 8, wherein biomarker 1 is sTNFR1; biomarker 2 is sVCAM1; and
biomarker 3 is sTNFR2.

11. The method of claim 1, wherein Alzheimer's disease comprises: normal cognition, mild cognitive impairment (MCI), or dementia.

12. A panel of biomarkers for use in determining progression of Alzheimer's disease in a CSF sample from a subject, wherein the biomarkers are sTNFR1, sTNFR2, and sVCAM1.

13. The panel of biomarkers of claim 11, further comprising biomarkers selected from the group consisting of: beta-amyloid 1-42 (Ab42); total tau (t-Tau); tau phosphorylated at threonine 181 (p-Tau₁₈₁); Transforming Growth Factor 1, 2, and 3 (TGFβ1, TGFβ2, TGFβ3); soluble Intercellular Adhesion Molecule 1 (sICAM1); Tumor Necrosis Factor α (TNFα); Interleukin 6 (IL)-6; IL-7; IL-9; IL-10; IL-12p40; IL-21; and Interferon Gamma-Induced Protein 10 (IP-10), or combinations thereof.
